(19) United States
(12) Patent Application Publication
(10) Pub. No.: US 2025/0359948 A1
(43) Pub. Date: Nov. 27, 2025

(54) MEDICAL DEVICE WRIST

(71) Applicant: Intuitive Surgical Operations, Inc., Sunnyvale, CA (US)

(72) Inventors: Erik NELSON, Durango, CO (US); Matthew A. WIXEY, Rochester, CT (US)

(73) Assignee: Intuitive Surgical Operations, Inc., Sunnyvale, CA (US)

(21) Appl. No.: 18/846,454

(22) PCT Filed: Mar. 8, 2023
(86) PCT No.: PCT/US2023/014818

§ 371 Date: Sep. 12, 2024

Related U.S. Application Data
(60) Provisional application No. 63/319,971, filed on Mar. 15, 2022.

(51) Int. Cl. A61B 34/30; B25J 9/10; B25J 17/02

(52) U.S. Cl. CPC A61B 34/30; B25J 9/1045; B25J 17/0241; A61B 2034/306

(57) ABSTRACT

A medical device includes a wrist link. a tool member, and a tension element. The wrist link includes a discrete first link piece and a discrete second link piece. The first link piece includes a first clevis ear and the second link piece includes a second clevis car. The second link piece is coupled to the first link piece to position the second clevis ear opposite the first clevis car and to define a tension element guide channel between the first link piece and the second link piece. The tool member is coupled to rotate between the first clevis ear and the second clevis car about a tool member rotation axis. The tension element is coupled to the tool member and extends from the tool member through the tension element guide channel. Tension on the tension element urges the tool member to rotate about the tool member rotation axis.

### CROSS-REFERENCE TO RELATED APPLICATIONS

This application claims benefit of priority to U.S. Provisional Application Ser. No. 63/319,971, entitled “Medical Device Wrist,” filed Mar. 15, 2022, which is incorporated herein by reference in its entirety.

### BACKGROUND

The embodiments described herein relate to medical devices, and more specifically to endoscopic tools. More particularly, the embodiments described herein relate to medical devices that include wrist mechanisms having one or more links constructed from multiple discrete pieces.

Known techniques for Minimally Invasive Surgery (MIS) employ instruments to manipulate tissue that can be either manually controlled or controlled via computer-assisted teleoperation. Many known MIS instruments include a therapeutic or diagnostic end effector (e.g., forceps, a cutting tool, or a cauterizing tool) mounted on a wrist mechanism at the distal end of a shaft. During an MIS procedure, the end effector, wrist mechanism, and the distal end of the shaft are inserted into a small incision or a natural orifice of a patient to position the end effector at a work site within the patient's body. The wrist mechanism can be used to change the end effector's orientation with reference to the shaft to perform the desired procedure at the work site. Known wrist mechanisms generally provide the desired mechanical degrees of freedom (DOFs) for movement of the end effector. For example, known wrist mechanisms are able to change the pitch and yaw orientation of the end effector with reference to the shaft's longitudinal axis. A wrist may optionally provide a roll DOF for the end effector with reference to the shaft, or an end effector roll DOF may be implemented by rolling the shaft, wrist, and end effector together as a unit. An end effector may optionally have additional mechanical DOFs, such as grip or knife blade motion. In some instances, wrist and end effector mechanical DOFs may be combined to provide various end effector control DOFs. For example, U.S. Pat. No. 5,792,135 (filed May 16, 1997) discloses a mechanism in which wrist and end effector grip mechanical DOFs are combined to provide an end effector yaw control DOF.

To enable the desired movement of the distal wrist mechanism and end effector, known instruments include cables that extend through the shaft of the instrument and that connect the wrist mechanism to a mechanical structure configured to move the cables to operate the wrist mechanism and end effector. For teleoperated systems, the mechanical structure is typically motor driven and is operably coupled to a computer processing system to provide a user interface for a clinical user (e.g., a surgeon) to control the instrument as a whole, as well as the instrument's components and functions.

Patients benefit from continual efforts to improve the effectiveness of MIS methods and devices. For example, reducing the size and/or the operating footprint of the shaft and wrist mechanism can allow for smaller entry incisions and reduced need for space at the surgical site, thereby reducing the negative effects of surgery, such as pain, scarring, and undesirable healing time. But producing small medical devices that implement the clinically desired functions for minimally invasive procedures can be challenging. Specifically, simply reducing the size of known wrist mechanisms by scaling down the components will not result in an effective solution because required component and material properties do not scale at relatively small physical dimensions. For example, efficient implementation of a wrist mechanism can be complicated because the cables must be carefully routed through the wrist mechanism to maintain cable tension throughout the range of motion of the wrist mechanism or end effector and to minimize the interactions (coupling effects) of motion about one rotation axis upon motion about another rotation axis. As another example, pulleys and/or contoured surfaces are generally needed to reduce cable friction, which permits operation without excessive forces being applied to the cables or other structures in the wrist mechanism. But increased localized forces that may result from smaller structures and cable bend radii (including smaller diameter cables and other wrist and end effector components) can result in undesirable lengthening (e.g., stretch or creep) of the cables during storage and use, reduced cable life, and the like.

Further. the wrist mechanism generally provides specific degrees of freedom for movement of the end effector. For example, for forceps or other grasping tools, the wrist may be able to change the end effector pitch, yaw, and grip orientations with reference to the instrument shaft. More degrees of freedom could be implemented through the wrist but would require additional actuation members (e.g., cables) in the wrist and shaft, and these additional members compete for the limited space that exists given the size restrictions required by MIS applications. Components needed to actuate other degrees of freedom, such as end effector roll or insertion/withdrawal through movement of the main tube, also compete for space at or in the shaft of the device.

A conventional architecture for a wrist mechanism in a manipulator-driven medical device uses cables pulled in and payed out by a capstan in the proximal mechanical structure and thereby rotate the portion of the wrist mechanism that is connected to the capstan via the cables. For example, a wrist mechanism can be operably coupled to three capstans-one each for rotations about a pitch axis, a yaw axis, and a grip axis. Each capstan can be controlled by using two cables that are attached to the capstan so that one side pays out cable while the other side pulls in an equal length of cable. With this architecture, three degrees of freedom require a total of six cables extending from the wrist mechanism proximally back along the length of the instrument's main shaft tube to the instrument's proximal mechanical structure. Efficient implementation of a wrist mechanism and proximal mechanical structure can be complicated because the cables must be carefully routed through the tool member, wrist mechanism, and proximal mechanical structure to maintain stability of the wrist throughout the range of motion of the wrist mechanism and to minimize the interactions (or coupling effects) of one rotation axis upon another.

In addition to the need to decrease the size and increase the performance of wrist devices, it is also desirable to develop low-cost instruments that are effectively disposable (i.e., that are intended for a single use only at an economic cost). With such instruments, each MIS procedure can be performed with a new, sterilized instrument, which eliminates cumbersome and expensive instrument reuse cleaning and sterilization procedures. Many current instrument designs are expensive to produce, however, and so these instruments undergo sterile reprocessing for use during multiple surgical procedures.

Additionally, to achieve the desired performance, known wrist mechanisms include many complex parts, including one or more clevises that define complex cable channels, pulleys, and in some cases, electronic components (for cautery instruments). Assembly of such known wrist mechanisms involves many complicated operations, which can further increase the cost of producing the wrist mechanism.

Thus, a need exists for improved wrist mechanisms that can be more easily assembled and include fewer parts, while still providing the desired performance.

Additionally, with smaller instruments, achieving the desired output force (e.g., for rotating the end effector about a pitch axis or rotating cutting blades about a grip axis) can be challenging due to the reduced space. Thus, a need also exists for wrist mechanisms with improved cable channels to optimize the output forces in several degrees of freedom.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In some embodiments, a medical device includes a wrist link, a tool member, and a tension element. The wrist link includes a discrete first link piece and a discrete second link piece. The first link piece includes a first clevis ear and the second link piece includes a second clevis ear. The second link piece is coupled to the first link piece to position the second clevis ear opposite the first clevis ear and to define a tension element guide channel between the first link piece and the second link piece. The tool member is coupled to rotate between the first clevis ear and the second clevis ear about a tool member rotation axis. The tension element is coupled to the tool member and extends from the tool member through the tension element guide channel. Tension on the tension element urges the tool member to rotate about the tool member rotation axis.

In some embodiments, the first link piece is substantially identical to the second link piece. In some embodiments, a configuration of the first link piece is the same as a configuration of the second link piece.

In some embodiments, the wrist link is a distal wrist link and the medical device includes a proximal wrist link and a connector link. The connector link includes a distal end and a proximal end. The distal wrist link is coupled to the distal end of the connector link and the proximal wrist link is coupled to the proximal end of the connector link. The distal wrist link rotates with reference to the connector link about a distal connector link rotation axis. The connector link rotates with reference to the proximal wrist link about a proximal connector link rotation axis. The distal wrist link is in rolling contact with the proximal wrist link as the distal wrist link rotates with reference to the proximal wrist link.

In some embodiments, the first link piece includes a first connector link receptacle and the second link piece includes a second connector link receptacle. The second link piece is coupled to the first link piece to position the second connector link receptacle opposite the first connector link receptacle. The distal end of the connector link is rotatably secured to the distal wrist link between the first connector link receptacle and the second connector link receptacle. In some embodiments, a first protrusion of the distal end of the connector link is within the first connector link receptacle and a second protrusion of the distal end of the connector link is within the second connector link receptacle.

In some embodiments, a longitudinal axis is defined between the distal and proximal ends of the connector link. The proximal wrist link includes a connector link receptacle that accepts insertion of the proximal end of the connector link at a first orientation of the connector link about the longitudinal axis of the connector link. The connector link receptacle resists withdrawal of the proximal end of the connector link at a second orientation of the connector link about the longitudinal axis of the connector link.

In some embodiments, the proximal wrist link includes a discrete third link piece and a discrete fourth link piece. The third link piece includes a third connector link receptacle and the fourth link piece includes a fourth connector link receptacle. The fourth link piece is coupled to the third link piece to position the fourth connector link receptacle opposite the third connector link receptacle. The proximal end of the connector link is rotatably secured to the proximal wrist link between the third connector link receptacle and the fourth connector link receptacle.

In some embodiments. the second link piece is coupled to the first link piece by any of an adhesive joint, a weld joint, or a mechanical fastener.

In some embodiments, a medical device includes a first link piece, a second link piece discrete from the first link piece, a tool member, and a connector link. The first link piece includes a distal end portion and a proximal end portion, and the distal end portion includes a first clevis ear, and the proximal end portion includes a first connector. The second link piece includes a distal end portion and a proximal end portion, and the distal end portion includes a second clevis ear, and the proximal end portion includes a second connector. The second link piece is coupled to the first link piece to form a wrist link and to position the second clevis ear opposite the first clevis ear and to position the second connector opposite the first connector. The tool member is coupled to rotate between the first clevis ear and the second clevis ear about a tool member rotation axis. The connector link is coupled to rotate between the first connector and second connector about a connector link rotation axis.

In some embodiments, the wrist link is a distal wrist link and the connector link includes a distal end and a proximal end. The distal end of the connector link is rotatably coupled between the first connector and the second connector. The medical device further includes a proximal wrist link. The proximal end of the connector link is rotatably coupled to the proximal wrist link.

In some embodiments, the medical device includes a tension element. The second link piece is coupled to the first link piece to define a tension element guide channel between the first link piece and the second link piece. The tension element is coupled to the tool member and extends from the tool member through the tension element guide channel. When tension is exerted on the tension element, it urges the tool member to rotate about the tool member rotation axis.

In some embodiments, the tool member rotation axis is perpendicular to the connector link rotation axis.

In some embodiments, a medical device includes a distal wrist link, a proximal wrist link, and a connector link. The distal wrist link includes a first tool support and a second tool support opposite the first tool support and configured to be rotatably coupled to a tool member. The distal wrist link includes a distal connector link receptacle and the proximal wrist link includes a proximal connector link receptacle. The connector link includes a distal end and a proximal end, and a longitudinal axis is defined through the distal and proximal ends of the connector link. The distal end of the connector link is coupled within the distal connector link receptacle, and the distal wrist link is rotatable with reference to the connector link about a distal connector link rotation axis. The proximal end of the connector link is coupled within the proximal connector link receptacle, and the proximal wrist link is rotatable with reference to the connector link about a proximal connector link rotation axis. At least one of the proximal connector link receptacle or the distal connector link receptacle is configured to accept insertion of the connector link at a first orientation of the connector link about the longitudinal axis of the connector link. At least one of the proximal connector link receptacle or the distal connector link receptacle is configured to resist withdrawal of the connector link at a second orientation of the connector link about the longitudinal axis of the connector link.

In some embodiments, the distal wrist link is in rolling contact with the proximal wrist link as the distal wrist link rotates with reference to the proximal wrist link.

In some embodiments, the proximal connector link receptacle is configured to accept insertion of the connector link at the first orientation. The proximal connector link receptacle or the distal connector link receptacle is configured to resist withdrawal of the connector link at the second orientation. An end surface of the proximal wrist link defines an insertion opening into the proximal connector link receptacle. A shape of the insertion opening taken within a plane normal to the longitudinal axis defines an insertion major axis that is aligned with the first orientation of the connector link about the longitudinal axis of the connector link.

In some embodiments, the distal wrist link includes a discrete first link piece and a discrete second link piece. The first link piece includes the first tool support and the second link piece includes the second tool support. The second link piece is coupled to the first link piece to position the second tool support opposite the first tool support.

In some embodiments, the second link piece is coupled to the first link piece to define a tension element guide channel between the first link piece and the second link piece. The medical device includes a tension element coupled to the tool member and extending from the tool member through the tension element guide channel. Tension on the tension element urges the tool member to rotate about the tool member rotation axis.

In some embodiments, methods of assembling a medical device are disclosed herein. The medical device includes a first link piece, a second link piece, a tool member, a pin, and a tension element. The first link piece includes a first clevis ear and a first guide channel. The second link piece includes a second clevis ear and a second guide channel. The pin includes a first end portion, a second end portion, and a central portion. The tool member is rotatably coupled about the central portion of the pin, and the tension element is coupled to the tool member. The method of assembly includes inserting the first end portion of the pin into the first clevis ear. The second end portion of the pin is inserted into the second clevis ear. A portion of the tension element is placed into at least one of the first guide channel or the second guide channel. The method includes positioning the second link piece over the first link piece so that the second clevis ear is opposite the first clevis ear, and coupling the second link piece to the first link piece to form a wrist link.

In some embodiments, a medical device includes a proximal wrist link, a distal wrist link wrist link, a tool member, and a tension element. The proximal wrist link includes a proximal end portion and a distal end portion, and defines a proximal tension element guide channel. The distal wrist link includes a proximal end portion and a distal end portion, and defines a distal tension element guide channel. The proximal end portion of the distal wrist link is coupled to the distal end portion of the proximal wrist link such that the distal wrist link rotates with reference to the proximal wrist link about a wrist rotation axis. A longitudinal center line is defined between the proximal end portion of the proximal wrist link and the distal end portion of the distal wrist link. A first distal wrist link plane is defined normal to the longitudinal center line and at a first position within the distal wrist link along the longitudinal center line, and a second distal wrist link plane is defined normal to the longitudinal center line and at a second position within the distal wrist link along the longitudinal center line. The tension element is coupled to the tool member and extends from the tool member through the distal tension element guide channel and through the proximal tension element guide channel. Tension on the tension element urges at least one of the distal wrist link to rotate about the wrist rotation axis or the tool member to rotate about a tool member rotation axis. A first central portion of the tension element is spaced a first X distance from the longitudinal center line along a first dimension within the distal wrist link entry plane and a first Y distance from the longitudinal center line along a second dimension within distal wrist link entry plane within the distal wrist link entry plane. A second central portion of the tension element is spaced a second X distance from the longitudinal center line along the first dimension within the distal wrist link exit plane and a second Y distance from the longitudinal center line along the second dimension within the distal wrist link exit plane. The first X distance is greater than the second X distance and the first Y distance is less than the second Y distance.

Other medical instruments, related components, medical device systems, and/or methods according to embodiments will be or become apparent to one with skill in the art upon review of the following drawings and detailed description. It is intended that all such additional medical devices, related components, medical device systems, and/or methods included within this description be within the scope of this disclosure.

### BRIEF DESCRIPTION OF THE DRAWINGS

FIG. 1 is a plan view of a minimally invasive teleoperated medical system according to an embodiment being used to perform a medical procedure such as surgery.

FIG. 2 is a perspective view of a user control console of the minimally invasive teleoperated surgery system shown in FIG. 1.

FIG. 3 is a perspective view of an optional auxiliary unit of the minimally invasive teleoperated surgery system shown in FIG. 1.

FIG. 4 is a front view of a manipulator unit, including a plurality of instruments, of the minimally invasive teleoperated surgery system shown in FIG. 1.

FIG. 5A is a diagrammatic exploded view of a medical device according to an embodiment.

FIG. 5B is a perspective exploded view of a portion of the medical device shown in FIG. 5A.

FIG. 5C is a rear side view and FIG. 5D is a top view of the medical device shown in FIG. 5A.

FIG. 6A is a diagrammatic exploded view of a medical device according to an embodiment.

FIG. 6B is a rear side view and FIG. 6C is a top view of the medical device shown in FIG. 6A.

FIG. 7A is a diagrammatic side view of a medical device according to an embodiment.

FIGS. 7B and 7C are a top view of the medical device shown in FIG. 7A in a first orientation (FIG. 7B) and a second orientation (FIG. 7C).

FIG. 7D is a perspective view of a connector link of the medical device shown in 7A.

FIG. 7E is a cross-sectional view of a portion of the medical device taken along line E-E in FIG. 7B. FIG. 7F is a cross-sectional view of a portion of the medical device taken along line F-F in FIG. 7B.

FIG. 8A is diagrammatic top view of a medical device according to an embodiment, and FIG. 8B is a diagrammatic side view of the medical device shown in FIG. 8A.

FIG. 9 is a perspective view of a medical device according to an embodiment.

FIG. 10 is a perspective view of a distal end portion of the medical device of FIG. 9.

FIG. 11 is an exploded perspective view of select components of the distal end portion of the medical device of FIG. 9.

FIG. 12 is a top view and FIG. 13 is a side view of the distal end portion of the medical device of FIG. 9.

FIG. 14 is a perspective view and FIG. 15 is a front view (taken in plane P1 shown in FIG. 12) of a proximal wrist link of the medical device of FIG. 9.

FIG. 16A is a perspective view of the proximal wrist link and the connector link of the medical device of FIG. 9, shown in a first configuration.

FIG. 16B is a top view of the proximal wrist link and the connector link of the medical device of FIG. 9, shown in the first configuration.

FIG. 17A is a perspective view of the proximal wrist link and the connector link of the medical device of FIG. 9, shown in a second configuration.

FIG. 17B is a top view of the proximal wrist link and the connector link of the medical device of FIG. 9, shown in an intermediate configuration between the first configuration and the second configuration.

FIG. 17C is a perspective view of the proximal wrist link and the connector link of the medical device of FIG. 9, shown in a second configuration.

FIG. 18 is a top perspective view and FIG. 19 is a bottom perspective view of a distal wrist link of the medical device of FIG. 9.

FIG. 20 is a partially exploded perspective view of the medical device of FIG. 9 showing the cables routed through the distal wrist link and coupled to the end effector.

FIG. 21 is a perspective view of the medical device of FIG. 9 with a link piece removed to show the cables routed through the distal wrist link and coupled to the end effector.

FIG. 22 is a front view of the distal wrist link of the medical device of FIG. 9 taken in plane P2 shown in FIG. 21 to show the position of the cable in plane P2.

FIG. 23 is a front view of the distal wrist link of the medical device of FIG. 9 taken in plane P3 shown in FIG. 21 to show the position of the cable in plane P3.

FIG. 24 is a perspective view of a distal end portion of the medical device where each of the proximal link and the distal link are constructed from discrete link pieces.

FIG. 25 is an exploded perspective view of select components of the distal end portion of the medical device of FIG. 24.

FIG. 26 is a top view and FIG. 27 is a side view of the distal end portion of the medical device of FIG. 24.

FIG. 28 is a top perspective view and FIG. 29 is a bottom perspective view of a proximal wrist link of the medical device of FIG. 244.

FIG. 30 is a top perspective view and FIG. 31 is a bottom perspective view of a distal wrist link of the medical device of FIG. 24.

FIG. 32 is a side view of the medical device of FIG. 24 with portions of the link pieces in transparent to show the cable routed through the wrist assembly and coupled to the end effector.

FIG. 33 is a perspective view of the medical device of FIG. 24 with a link piece removed to show the cables routed through the wrist assembly and coupled to the end effector.

FIG. 34 is a top view of the medical device of FIG. 24 with the wrist assembly in a rotated orientation and with a link piece removed to show the cables routed through the wrist assembly and coupled to the end effector.

FIG. 35 is a front view (taken in plane P1 shown in FIG. 34) of a proximal wrist link of the medical device of FIG. 24.

FIG. 36 is a front view of the distal wrist link of the medical device of FIG. 24 taken in plane P2 shown in FIG. 33 to show the position of the cable in plane P2.

FIG. 37 is a front view of the distal wrist link of the medical device of FIG. 24 taken in plane P3 shown in FIG. 33 to show the position of the cable in plane P3.

FIG. 38 is a flow chart of a method of assembling a medical device according to an embodiment.

### DETAILED DESCRIPTION

The embodiments described herein can advantageously be used in a wide variety of grasping, cutting, and manipulating operations associated with minimally invasive surgery. In some embodiments, an end effector of the medical device can move with reference to the main body of the instrument in three mechanical DOFs, e.g., pitch, yaw, and roll (shaft roll). There may also be one or more mechanical DOFs in the end effector itself. e.g., two jaws, each rotating with reference to a clevis (2 DOFs) and a distal clevis that rotates with reference to a proximal clevis (one DOF).

The medical devices of the present application enable motion in three degrees of freedom (e.g., about a pitch axis, a yaw axis, and a grip axis) using only four cables, thereby reducing the total number of cables required, reducing the space required within the shaft and wrist, reducing overall cost, and enables further miniaturization of the wrist and shaft assemblies to promote MIS procedures. Moreover, the medical devices described herein can include clevises or wrist links that are assembled by coupling two separate pieces together. This arrangement can allow for improvements in manufacturing, for example, by allowing the cables to be placed within one or more cable channels before the assembly of the wrist. Such improvements can reduce costs, thereby facilitating a single-use device. The medical devices described herein can have a reduced number of parts unique parts, which can also reduce cost.

As described herein, in some embodiments, a medical device includes a first link piece and a second link piece that is discrete from the first link piece. The first link piece includes a first clevis ear and a first connector. The second link piece includes a second clevis ear and a second connector. The two link pieces can be coupled together to form a wrist link that has the second clevis ear opposite the first clevis ear and the second connector opposite the first connector. A tool member can be coupled to rotate between the two clevis ears, and a connector link can be coupled to the two connectors.

Medical devices described herein can include one or more cables (which function as tension elements) that are made of a polymer material and that can be routed through a wrist along one or more cable channels. The cable channels can be nonlinear and can be shaped such that the cable is spaced a first distance (along a first dimension) from a center line of the wrist at a first location. The first distance can be selected to maximize the torque applied by the cable at that point. The can be spaced a second distance (along a second dimension) from the center line at a second location. The second distance can be selected to maximize the torque applied by the cable at that point. The cable channel can be shaped such that the two distances are maintained within an overall footprint (or boundary) of the device. This arrangement can allow for the desired torque performance of the wrist while facilitating miniaturization of the wrist.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site, and the word “proximal” refers to a direction away from the work site. Thus, for example, the end of a medical device that is closest to the target tissue would be the distal end of the medical device, and the end opposite the distal end (i.e., the end manipulated by the user or coupled to the actuation shaft) would be the proximal end of the medical device.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial positions and orientations. The combination of a body's position and orientation define the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, medical instrument, and variants thereof, can be interchangeably used.

Aspects of the invention are described primarily in terms of an implementation using a da Vinci® surgical system, commercialized by Intuitive Surgical, Inc. of Sunnyvale, California. Examples of such surgical systems are the da Vinci Xi® surgical system (Model IS4000), da Vinci X® Surgical System (Model IS4200), and the da Vinci Si® surgical system (Model IS3000). Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations on da Vinci® surgical systems (e.g., the Model IS4000, the Model IS3000, the Model IS2000, the Model IS1200, the Model SP1099) are merely presented as examples, and they are not to be considered as limiting the scope of the inventive aspects disclosed herein. As applicable, inventive aspects may be embodied and implemented in both relatively smaller, hand-held, hand-operated devices that are not mechanically grounded in a world reference frame and relatively larger systems that have additional mechanical support that is grounded in a world reference frame.

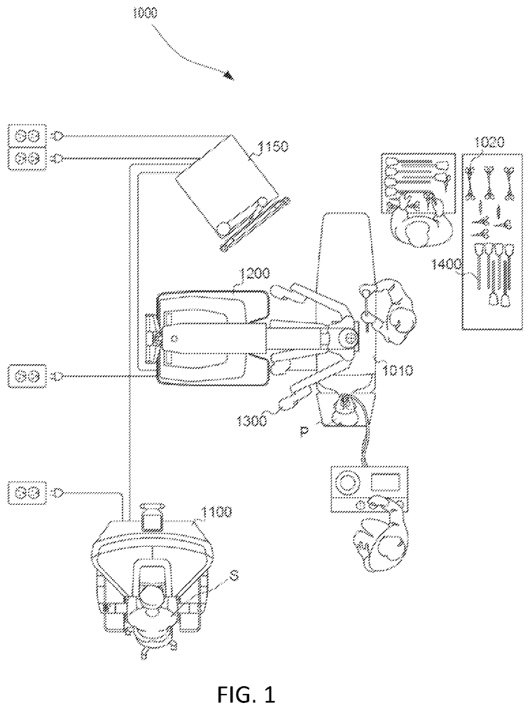

FIG. 1 is a plan view illustration of a teleoperated surgical system 1000 that operates with at least partial computer assistance (a “telesurgical system”). Both telesurgical system 1000 and its components are considered medical devices. Telesurgical system 1000 is a Minimally Invasive Robotic Surgical (MIRS) system used for performing a minimally invasive diagnostic or surgical procedure on a Patient P who is lying on an Operating table 1010. The system can have any number of components, such as a user control unit 1100 for use by a surgeon or other skilled clinician S during the procedure. The MIRS system 1000 can further include a manipulator unit 1200 (popularly referred to as a surgical robot) and an optional auxiliary equipment unit 1150. The manipulator unit 1200 can include an arm assembly 1300 and a surgical instrument tool assembly removably coupled to the arm assembly. The manipulator unit 1200 can manipulate at least one removably coupled instrument 1400 through a minimally invasive incision in the body or natural orifice of the patient P while the surgeon S views the surgical site and controls movement of the instrument 1400 through control unit 1100. An image of the surgical site is obtained by an endoscope (not shown), such as a stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the MIRS 1000.

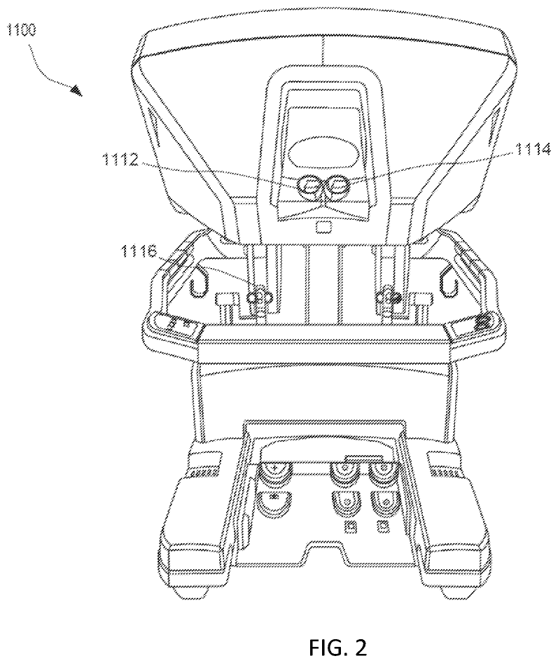

FIG. 2 is a perspective view of the control unit 1100. The user control unit 1100 includes a left eye display 1112 and a right eye display 1114 for presenting the surgeon S with a coordinated stereoscopic view of the surgical site that enables depth perception. The user control unit 1100 further includes one or more input control devices 1116, which in turn cause the manipulator unit 1200 (shown in FIG. 1) to manipulate one or more tools. The input control devices 1116 provide at least the same degrees of freedom as instruments 1400 with which they are associated to provide the surgeon S with telepresence, or the perception that the input control devices 1116 are integral with (or are directly connected to) the instruments 1400. In this manner, the user control unit 1100 provides the surgeon S with a strong sense of directly controlling the instruments 1400. To this end, position, force, strain, or tactile feedback sensors (not shown) or any combination of such sensations, from the instruments 1400 back to the surgeon's hand or hands through the one or more input control devices 1116.

The user control unit 1100 is shown in FIG. 1 as being in the same room as the patient so that the surgeon S can directly monitor the procedure, be physically present if necessary, and speak to an assistant directly rather than over the telephone or other communication medium. In other embodiments, however, the user control unit 1100 and the surgeon S can be in a different room, a completely different building, or other location remote from the patient, allowing for remote surgical procedures.

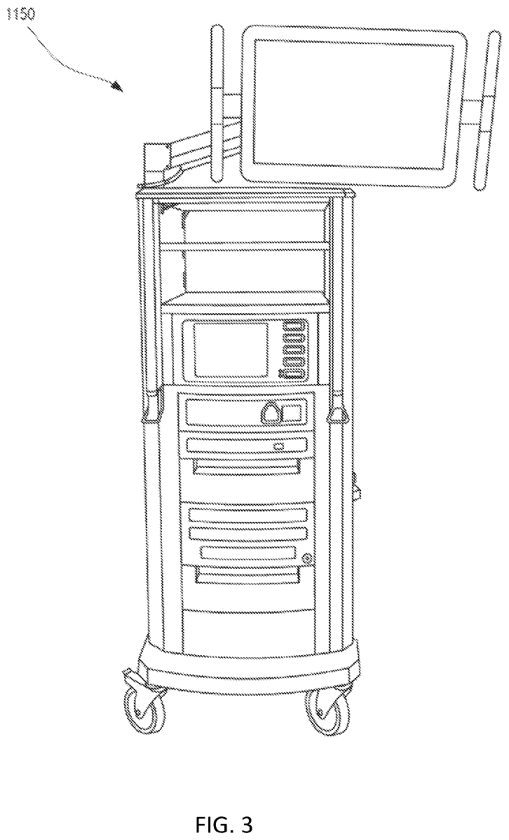

FIG. 3 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally (e.g., on the unit 1150 itself as shown, on a wall-mounted display) and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

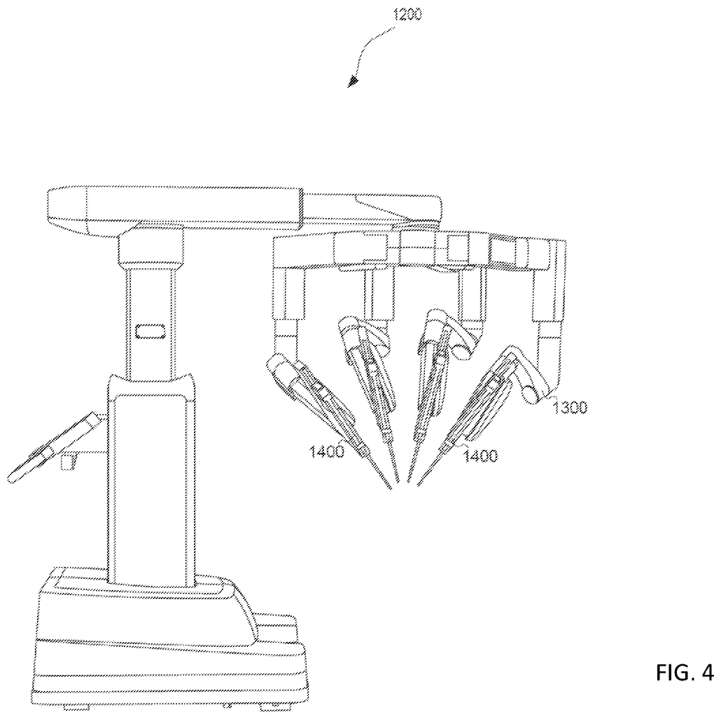

FIG. 4 shows a front perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient P in a manner such that a center of motion remote from the manipulator and typically located at a position along the instrument shaft is maintained at the incision or orifice by either kinematic mechanical or software constraints. In this manner, the incision size can be minimized.

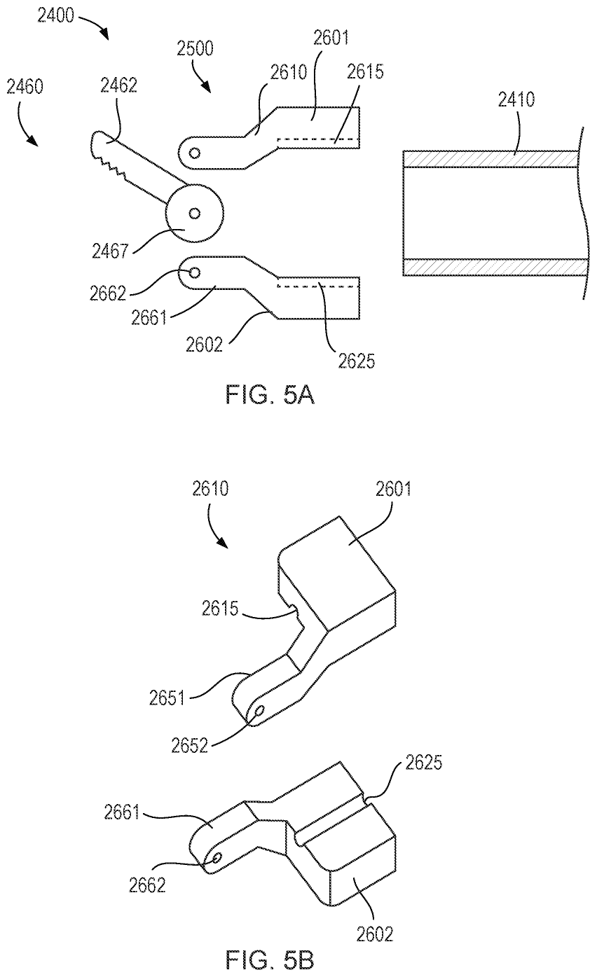

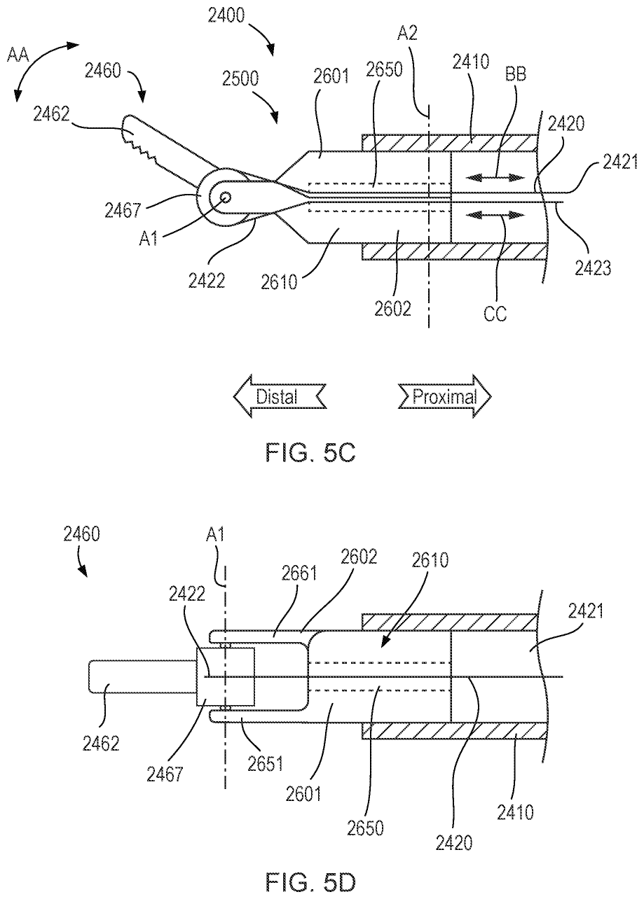

FIGS. 5A-5D are schematic illustrations of a portion of a medical device 2400 according to an embodiment. In some embodiments, the medical device 2400 or any of the components therein are optionally parts of an instrument of a surgical system that performs surgical procedures, and which can include a manipulator unit, a series of kinematic linkages, a series of cannulas, or the like. The medical device 2400 (and any of the instruments described herein) can be used in any suitable surgical system, such as the MIRS system 1000 shown and described above. The medical device 2400 includes a shaft 2410, a wrist assembly 2500 including at least one wrist link 2610, an end effector 2460, and a tension element 2420 (which can be a cable). Although only one tension element 2420 and one tool member 2462 are shown, one or more additional tension elements or one or more additional tool members can be included. The medical device 2400 is configured such that movement of the tension element 2420 produces movement of the wrist assembly 2500 (similar to the movement described below with reference to the medical device 3400), movement of the tool member 2462 (as illustrated in FIG. 5C), or both movement of the wrist assembly 2500 and movement of the tool member 2462.

The wrist assembly 2500 includes at least one wrist link 2610 that includes a discrete first link piece 2601 and a discrete second link piece 2602 (see FIG. 5B). Similarly stated, the first link piece 2601 is separate from the second link piece 2602. The first link piece 2601 and the second link piece 2602 are constructed as separate pieces and are later coupled together (as described herein) to form the wrist link 2610. By forming the wrist link 2610 from two discrete pieces, the method of assembly of the device 2400 can be made more efficient than that for a device with a monolithically constructed wrist link. For example, as described herein, the tension element 2420 can be placed into a tension element channel before the second link piece 2602 is coupled to the first link piece 2601, thereby eliminating the need to pass loose ends of the tension element through an enclosed channel. The second link piece 2602 can be coupled to the first link piece 2601 by any suitable mechanism (e.g., an adhesive joint, a weld joint, or a mechanical fastener).

As shown in FIGS. 5A and 5B, the first link piece 2601 includes a first clevis ear 2651 that defines an opening 2652. The first link piece 2601 also defines a channel 2615. The second link piece 2602 includes a second clevis ear 2661 that defines an opening 2662. The second link piece 2602 also defines a channel 2625. The channel 2615 of the first link piece 2601 and the channel 2625 of the second link piece 2602 form a tension element guide channel 2650 when the second link piece 2602 is coupled to the first link piece 2601. The tension element guide channel 2650 can have any suitable size, shape, or contour to provide a desired path for the tension element 2420 to pass therethrough. For example, the tension element guide channel 2650 can be shaped to reduce sharp bends, which can reduce friction losses when the tension element 2420 is moved within the tension element guide channel 2450. As another example, in some embodiments, the tension element guide channel 2650 can be shaped to ensure that the tension element 2420 is routed to the end effector in a manner that will produce the desired offset distance between the distal end portion 2422 of the tension element 2420 and the tool member rotation axis A1. In this manner, the magnitude of torque applied to the tool member 2462 (for a given amount of tension applied to the tension element) can be maximized. In some embodiments, the tension element guide channel 2650 can be shaped to produce the desired offset distance between tension element 2420 and the wrist rotation axis A2. For example, although FIG. 5D shows the tension element 2420 being aligned with the wrist rotation axis A2 in the top view plane, in other embodiments, the tension element guide channel 2650 can be curved to produce an offset distance (which will produce or increase a torque that the tension element 2420 can apply to the wrist link 2610 about the wrist rotation axis A2). Thus, the tension element guide channel 2650 can include any of the shapes or features as described below with reference to the medical devices 6400 and 7400.

As shown, when the second link piece 2602 is coupled to the first link piece 2601, the second clevis ear 2661 is opposite the first clevis ear 2651. Thus, the openings 2652 and 2662 are aligned such that a pin (not shown) or other structure can be coupled between the first clevis ear 2651 and the second clevis ear 2661 to allow rotation of the tool member 2462 about a tool member rotation axis A1. Similarly stated, when the second link piece 2602 is coupled to the first link piece 2601, the second clevis ear 2661 is aligned with the first clevis ear 2651 to define the tool member rotation axis A1.

In some embodiments, the first link piece 2601 and the second link piece 2602 can have the same configuration. Similarly stated, in some embodiments, the first link piece 2601 and the second link piece 2602 can be substantially identical pieces. In this manner, the number of unique parts included within the wrist assembly 2500 can be reduced, which further improves manufacturability (e.g., by reducing the number of parts and also reduce potential errors associated with coupling improper parts together). Moreover, in some embodiments, either the first link piece 2601 or the second link piece 2602 (or both) can be formed in part with an electrically conductive material. In this manner, either the first link piece 2601 or the second link piece 2602 (or both) can be a portion of an electrical circuit to deliver energy to the tool member 2462.

As shown, the wrist link 2610 is coupled to the shaft 2410. The shaft 2410 (and any of the shafts described herein) can include or be coupled to any suitable components, such as an inner shaft, insulation portions, spacers, and seals, or the like. The shaft 2410 can be formed, for example, with an electrically conductive material such as stainless steel. In some embodiments, the shaft 2410 (and any of the shafts described herein) can be similar to the shafts (and shaft assemblies) shown and described in copending U.S. Provisional Patent Application Ser. No. 63/294,103, entitled “Surgical Instrument Including Electrical and Fluid Isolation Features,” the disclosure of which is incorporated herein by reference in its entirety.

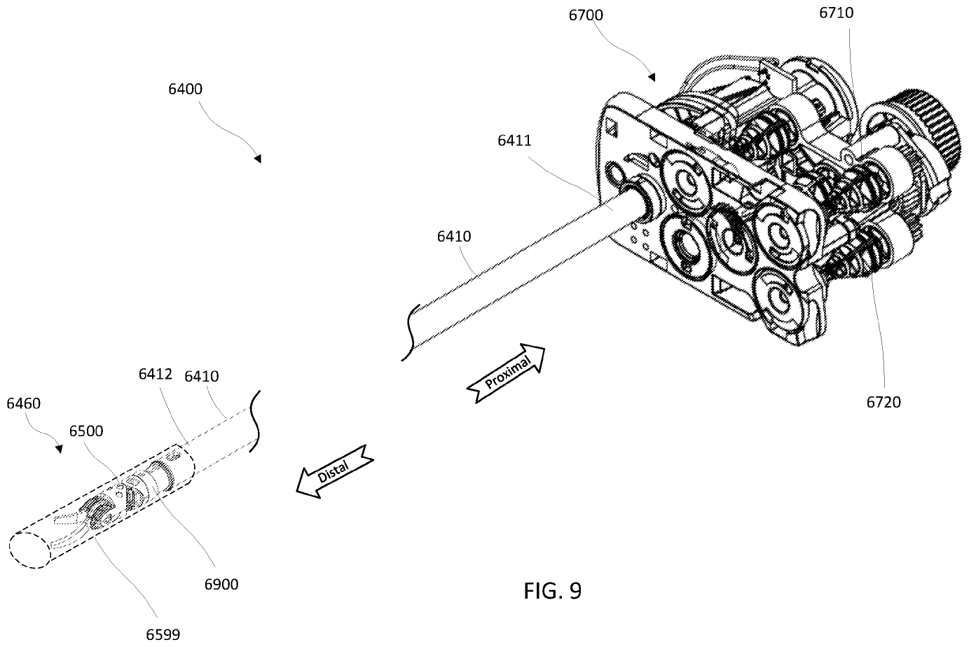

The wrist link 2610 is coupled to the shaft 2410 via any suitable mechanism, such as by welding, interference fit, adhesive, etc. In some embodiments, the wrist link 2610 can coupled to the shaft 2410 via another wrist link or connector link (not shown, but which can be similar to the connector link 3580 shown and described below) so that the wrist link 2610 can rotate about a wrist rotation axis A2 (which functions as a pitch axis; the term pitch is arbitrary). In such an embodiment, movement of the first proximal portion 2421 and the second proximal portion 2423 of the tension element 2420 can produce movement of the wrist link 2610 about the wrist rotation axis A2. An embodiment with a wrist assembly that includes multiple wrist links is shown and described below with reference to FIGS. 9-23.

The end effector 2460 includes at least one tool member 2462 that is coupled to rotate between the first clevis ear 2651 and the second clevis ear 2661 about a tool member rotation axis A1. More particularly, the tool member 2462 includes a contact portion and a pulley portion 2467. The contact portion is configured to engage or manipulate a target tissue during a surgical procedure. For example, in some embodiments, the contact portion can include an engagement surface that functions as a gripper, cutter, tissue manipulator, or the like. In other embodiments, the contact portion can be an energized portion of the tool member that is used for cauterization or electrosurgical procedures. The pulley portion 2467 is rotatably coupled to the first clevis ear 2651 and the second clevis ear 2661 by a pin (not shown) or other suitable mechanism that is coupled within the opening 2652 and 2662 and is aligned with the tool member rotation axis A1. The pulley portion 2467 includes a contact surface or other structure to which the distal end 2422 of the tension element 2420 is coupled. In this manner, when a tension is applied to the tension element 2420, the tension element 2420 urges the tool member 2462 to rotate about the tool member rotation axis A1. Similarly stated, tension applied causes movement of the first proximal portion 2421 (see that arrow BB in FIG. 5C) and/or the second proximal portion 2423 (see that arrow CC in FIG. 5C), which, in turn, produces movement of the tool member 2462 about tool member rotation A1 (which functions as the yaw axis; the term yaw is arbitrary), in a direction of arrows AA. In some embodiments, movement of the first proximal portion 2421 and the second proximal portion 2423 of the tension element 2420 can also produce movement of the wrist link 2610 about the wrist rotation axis A2. In some embodiments, the wrist rotation axis A2 is non-parallel to the tool member rotation axis A1.

Although only one tool member 2462 is shown, in other embodiments, the medical device 2400 can include two or more moving tool members that cooperatively perform gripping or shearing functions. Thus, the tool member rotation axis A1 can also function as a cutting axis as tool members rotate in opposition to each other as described in more detail below. Thus, in some embodiments, the medical device 2400, can provide at least three degrees of freedom (i.e., yaw motion about the tool member rotation axis A1, pitch rotation about the wrist rotation axis A2, and a cutting motion about the tool member rotation axis A1).

In some embodiments, the end effector 2460 and wrist assembly 2500 are operatively coupled to a mechanical structure (not shown, but which can be similar to the mechanical structure 6700 described below) that functions to receive one or more motor input forces or torques and mechanically transmit the received forces or torques (e.g., via the tension element 2420) to move an associated one or more components in the end effector 2460 and wrist assembly 2500. For example, one or more electric motors in manipulator unit 1200 (described above) provides an input to a mechanical structure, which in turn transmits the input via the tension element 2420 to move the tool member 2462 or the wrist link 2610 as described. The tension element 2420 can be routed along or through the shaft 2410 to couple the mechanical structure (not shown) at the proximal end of the shaft 2410 to the end effector 2460 at the distal end of the shaft 2410. More specifically, the tension element 2420 includes a first proximal portion 2421, a second proximal portion 2423 and a distal portion 2422. The first proximal portion 2421 and the second proximal portion 2423 extend through the tension element guide channel 2650, along (or through) the shaft 2410, and are coupled to the mechanical structure. The distal portion 2422 is coupled to the end effector 2460 (e.g., the pulley portion 2467 of the tool member 2462. In some embodiments, any of the tension elements described herein (including the tension elements 2420, 3420, 4420, 5420, 6420, 7420) can be a cable having a polymeric braided construction. With the tension element 2420 coupled to a mechanical structure (not shown) and to the end effector 2460, actuation at the mechanical structure causes the first proximal portion 2421 of the cable 2420 to move in a direction BB (e.g., proximally or distally depending on the direction of rotation), as shown in FIG. 5C. Similarly, actuation at the mechanical structure causes the second proximal portion 2423 of the cable 2420 to move in the direction CC (e.g., proximally or distally depending on the direction of rotation), as shown in FIG. 5C.

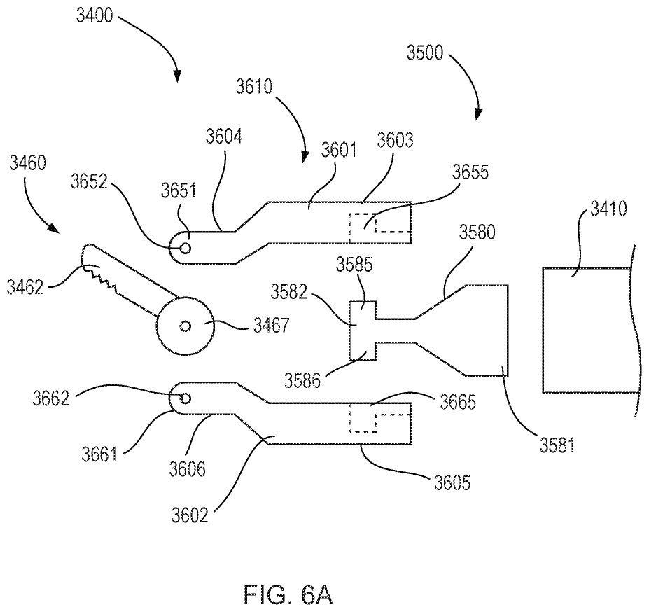

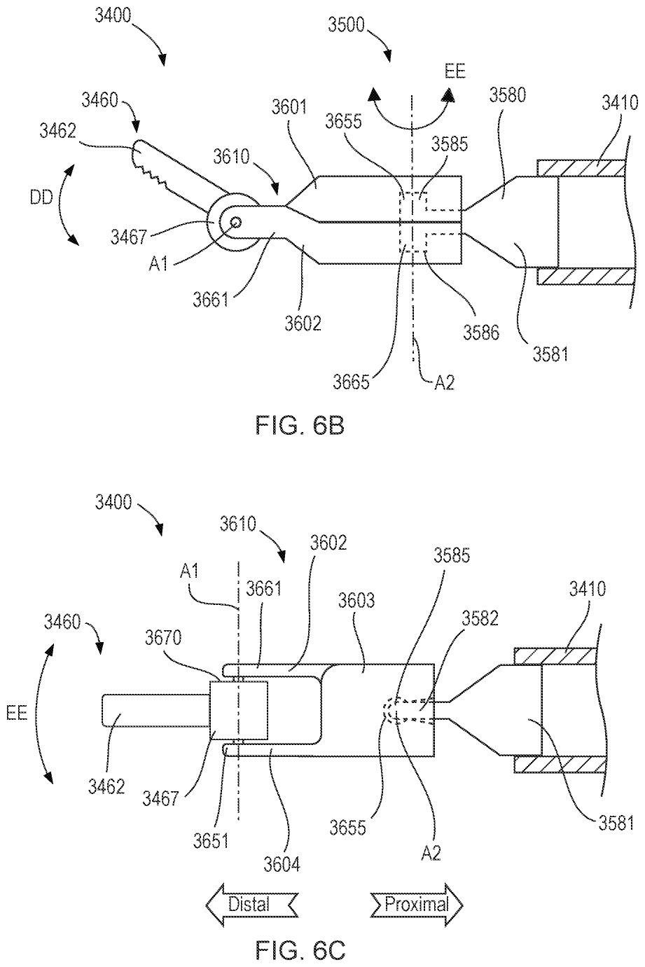

As described above, in some embodiments, a medical device can include a wrist assembly having multiple links to facilitate movement (e.g., rotation) about multiple different rotation axes. In some embodiments, a wrist assembly can include a link that is constructed from multiple discrete pieces that are coupled together to define multiple different coupling points with different axes of rotation. For example, FIGS. 6A-6C are schematic illustrations of a portion of a medical device 3400 according to an embodiment. In some embodiments, the medical device 3400 or any of the components therein are optionally parts of an instrument of a surgical system that performs surgical procedures, and which can include a manipulator unit, a series of kinematic linkages, a series of cannulas, or the like. The medical device 3400 (and any of the instruments described herein) can be used in any suitable surgical system, such as the MIRS system 1000 shown and described above. The medical device 3400 includes a shaft 3410, a wrist assembly 3500 including at least a wrist link 3610 and a connector link 3580, and an end effector 3460. Although only one tool member 3462 is shown, one or more additional tool members can be included. Moreover, although the medical device is not shown as including any tension elements, one or more tension elements (similar to the tension element 2420) can be included. In other embodiments, however, movement of the wrist or end effector can be caused by movement of any suitable actuator (not shown, but which can be, for example, a motor within the end effector, a hydraulic actuator, or movement of a tension member). Thus, movement of an actuator produces movement of the wrist assembly 3500, movement of the tool member 3462 (as illustrated in FIG. 6B), or both movement of the wrist assembly 3500 and movement of the tool member 3462.

The wrist assembly 3500 includes a wrist link 3610 and a connector link 3580 coupled to rotate relative to the wrist link 3610. The wrist link 3610 includes a discrete first link piece 3601 and a discrete second link piece 3602 (see FIG. 6A). Similarly stated, the first link piece 3601 and the second link piece 3602 are constructed as separate pieces and are later coupled together (as described herein) to form the wrist link 3610. The second link piece 3602 can be coupled to the first link piece 3601 by any suitable mechanism (e.g., an adhesive joint, a weld joint, or a mechanical fastener). As shown in FIGS. 6A and 6B, the first link piece 3601 includes a proximal end portion 3603 and a distal end portion 3604. The distal end portion 3604 includes a first clevis ear 3651 that defines an opening 3652. The proximal end portion 3603 includes a first connector 3655. The second link piece 3602 includes a proximal end portion 3605 and a distal end portion 3606. The distal end portion 3606 of the second link piece 3602 includes a second clevis ear 3661 that defines an opening 3662. The proximal end portion 3605 second link piece 3601 includes a second connector 3665.

The connector 3655 and the connector 3665 can be any suitable connectors to rotatably couple the connector link 3580 to the wrist link 3610, as described herein. For example, as shown the connector 3655 can include a receptacle (or volume) within which a protrusion 3583 of the connector link 3580 is rotatably coupled, and the connector 3665 can include a receptacle (or volume) within which a protrusion 3584 of the connector link 3580 is rotatably coupled. In other embodiments, however, either (or both) of the connector 3655 or the connector 3665 can include a protrusion that is coupled within a receptacle or bore of the connector link.

As shown, when the second link piece 3602 is coupled to the first link piece 3601, the second clevis ear 3661 is opposite the first clevis ear 3651. Thus, the openings 3652 and 3662 are aligned such that a pin 3670 can be coupled between the first clevis ear 3651 and the second clevis ear 3661 to allow rotation of the tool member 3462 about a tool member rotation axis A1. Similarly stated, when the second link piece 3602 is coupled to the first link piece 3601, the second clevis ear 3661 is aligned with the first clevis ear 3651 to define the tool member rotation axis A1. When the second link piece 3602 is coupled to the first link piece 3601, the second connector 3665 is opposite the first connector 3655. Thus, the connectors 3665 and 3655 are aligned such that a portion of the connector link 3580 (e.g., the protrusion 3583 and the protrusion 3584) can be coupled between the first connector 3655 and the second connector 3665 to allow rotation of the connector link 3580 about a connector link rotation axis A2. Similarly stated, when the second link piece 3602 is coupled to the first link piece 3601, the second connector 3665 is aligned with the first connector 3655 to define the connector link rotation axis A2. In some embodiments, the tool member rotation axis A1 is perpendicular to the connector link rotation axis A2.

In some embodiments, the medical device 3400 can include one or more tension elements that are coupled to a mechanical structure, and movement of the tension elements can produce the desired movement of the wrist assembly 3500, the tool member 3462, or both. Thus, although neither the first link piece 3601 nor the second link piece 3602 are shown as defining a channel that forms a tension element guide channel, in some embodiments, either (or both of) the first link piece 3601 or the second link piece 3602 can define one or more channels (not shown). Such channels can form one or more tension element guide channels when the first link piece 3601 is coupled to the second link piece 3602. Any tension element guide channels can have any suitable size, shape, or contour to provide a desired path for a tension element to pass therethrough, such as the shape of the tension element guide channels 6515 and 6615 described herein.

In some embodiments, the first link piece 3601 and the second link piece 3602 can have the same configuration. Similarly stated, in some embodiments, the first link piece 3601 and the second link piece 3602 can be substantially identical pieces. In this manner, the number of unique parts included within the wrist assembly 3500 can be reduced, which further improves manufacturability (e.g., by reducing the number of parts and also reduce potential errors associated with coupling improper parts together). Moreover, in some embodiments, either the first link piece 3601 or the second link piece 3602 (or both) can be formed in part with electrically conductive material. In this manner, either the first link piece 3601 or the second link piece 3602 (or both) can be a portion of an electrical circuit to deliver energy to the tool member 3462.

The connector link 3580 includes a proximal end portion 3581 and a distal end portion 3582. The distal end portion 3582 includes a first protrusion 3585 and a second protrusion 3586. The first protrusion 3585 is coupled to the first connector 3655 of the wrist link 3610. The second protrusion 3586 is coupled to the second connector 3665 of the wrist link 3610. As shown, the proximal end portion 3581 of the connector link 3580 is coupled to the shaft 3410. The shaft 3410 (and any of the shafts described herein) can include or be coupled to any suitable components, such as an inner shaft, insulation portions, spacers, and seals, or the like. The shaft 3410 can be similar to the shafts (and shaft assemblies) shown and described in copending U.S. Provisional Patent Application Ser. No. 63/294,103, entitled “Surgical Instrument Including Electrical and Fluid Isolation Features.” the disclosure of which is incorporated herein by reference in its entirety. The connector link 3580 is coupled to the shaft 3410 via any suitable mechanism, such as by welding, interference fit, adhesive, etc.

In some embodiments, the connector link 3580 can coupled to the shaft 3410 via another wrist link. For example, in some embodiments, the wrist link 3610 is a distal wrist link and the wrist assembly 3500 includes a proximal wrist link (not shown). In such embodiments, the proximal wrist link can be coupled to the shaft 3410 and the connector link 3580 can be rotatably coupled between the distal wrist link 3610 and the proximal wrist link. In such embodiments, the proximal end portion 3581 of the connector link 3580 can include protrusions (similar to the protrusions 3585 and 3586) or any other suitable connector that can be rotatably coupled to (or within) the proximal wrist link. An embodiment with a wrist assembly that includes a proximal wrist link, a distal wrist link, and a connector link coupled between the proximal and distal wrist links is shown and described below with reference to FIGS. 9-23.

The end effector 3460 includes at least one tool member 3462 that is coupled to rotate between the first clevis ear 3651 and the second clevis ear 3661 about a tool member rotation axis A1. More particularly, the tool member 3462 includes a contact portion and a pulley portion 3467. The contact portion is configured to engage or manipulate a target tissue during a surgical procedure. For example, in some embodiments, the contact portion can include an engagement surface that functions as a gripper, cutter, tissue manipulator, or the like. In other embodiments, the contact portion can be an energized portion of the tool member that is used for cauterization or electrosurgical procedures. The pulley portion 3467 is rotatably coupled to the first clevis ear 3651 and the second clevis ear 3661 by a pin 3670 or other suitable mechanism. The pulley portion 3467 includes a contact surface or other structure to which an actuator or tension element (not shown) can be coupled. In this manner, when actuated the tool member 3462 can rotate about the tool member rotation axis A1 (which functions as the yaw axis; the term yaw is arbitrary), in a direction of arrows DD. In some embodiments, the medical device 3400 can be actuated to produce movement of the wrist link 3610 relative to the connector link 3580 about the connector link rotation axis A2, in a direction of arrows EE. In some embodiments, the connector link rotation axis A2 is non-parallel to the connector link rotation axis A1.

Although only one tool member 3462 is shown, in other embodiments, the medical device 3400 can include two or more moving tool members that cooperatively perform gripping or shearing functions. Thus, the tool member rotation axis A1 can also function as a cutting axis as tool members rotate in opposition to each other as described in more detail below. Thus, in some embodiments, the medical device 3400, can provide at least three degrees of freedom (i.e., yaw motion about the tool member rotation axis A1, pitch rotation about the wrist rotation axis A2, and a cutting motion about the tool member rotation axis A1).

In some embodiments, the end effector 3460 and wrist assembly 3500 are operatively coupled to a mechanical structure (not shown, but which can be similar to the mechanical structure 6700 described below) that functions to receive one or more motor input forces or torques and mechanically transmit the received forces or torques (e.g., via a tension element or other suitable mechanism) to move an associated one or more components in the end effector 3460 and wrist assembly 3500.

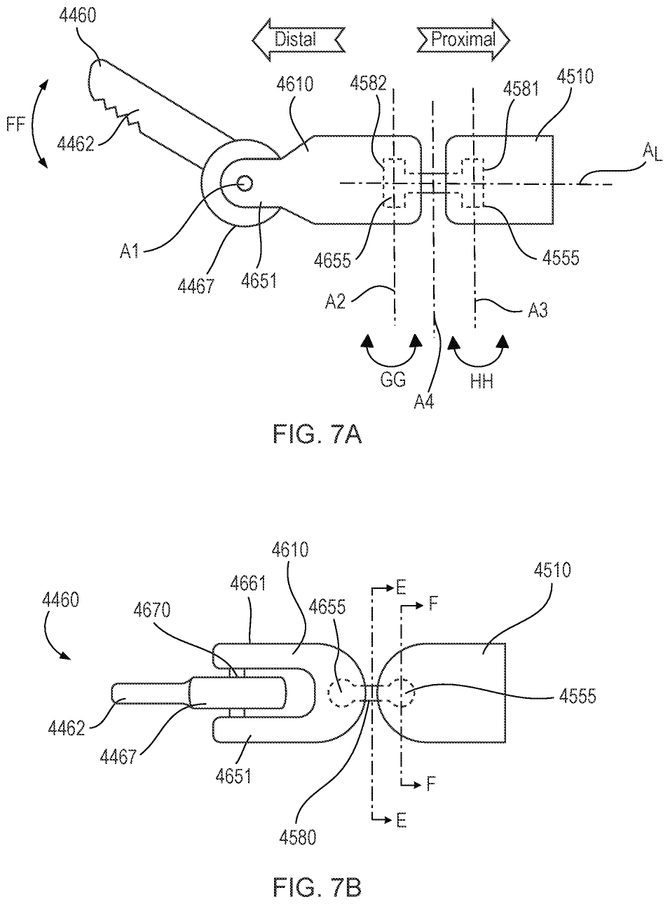

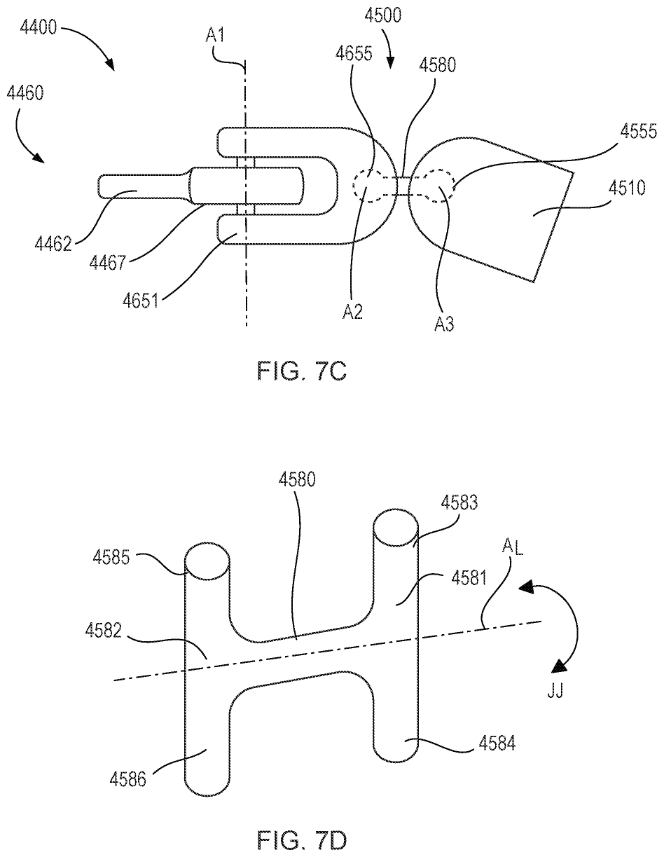

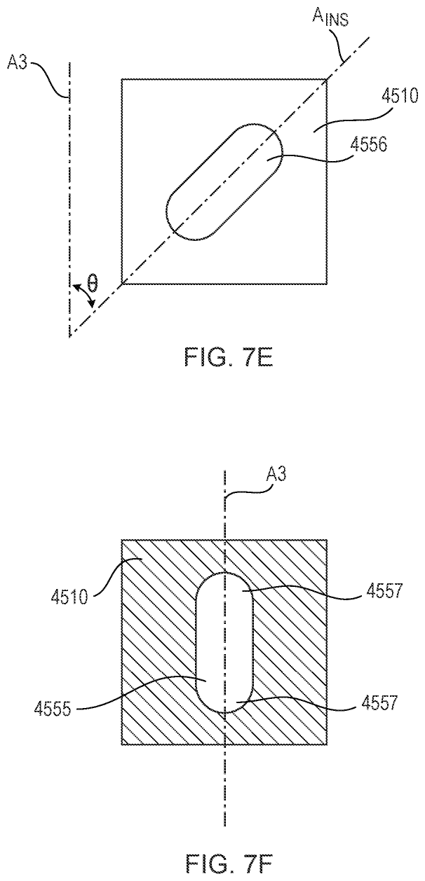

As described above, in some embodiments, a medical device can include a wrist assembly having a distal wrist link, a proximal wrist link, and a connector link rotatably coupled between the distal wrist link and the proximal wrist link. In some embodiments, either, both, or neither of the distal wrist link or the proximal wrist link can be constructed from two (or more) discrete link pieces as described above with reference to the wrist link 2610 and the wrist link 3610. In some embodiments, either or both of the distal wrist link and the proximal wrist link can include one or more connectors to which the connector link can be rotatably coupled. Moreover, in such embodiments, the connectors can be configured to accept insertion of a portion of the connector link when the connector link is in a first orientation and resist withdrawal (or removal) of the connector link when the connector link is in a second orientation. Similarly stated, in some embodiments, the connector link can be rotated (or otherwise undergo a change in orientation) to lock the connector link into the connector of the wrist link. Such an arrangement can allow for streamlined manufacturing processes. As one example, FIGS. 7A-7F are schematic illustrations of a portion of a medical device 4400 according to an embodiment. In some embodiments, the medical device 4400 or any of the components therein are optionally parts of an instrument of a surgical system that performs surgical procedures, and which can include a manipulator unit, a series of kinematic linkages, a series of cannulas, or the like. The medical device 4400 (and any of the instruments described herein) can be used in any suitable surgical system, such as the MIRS system 1000 shown and described above. The medical device 4400 includes a wrist assembly 4500 and an end effector 4460. Although only one tool member 4462 is shown, one or more additional tool members can be included. Moreover, although the medical device is not shown as including any tension elements, one or more tension elements (similar to the tension element 2420) can be included. In other embodiments, however, movement of the wrist or end effector can be caused by movement of any suitable actuator (not shown, but which can be, for example, a motor within the end effector, a hydraulic actuator, or movement of a tension member). Thus, movement of an actuator produces movement of the wrist assembly 4500 (see FIG. 7C), movement of the tool member 4462 (see FIG. 7A), or both movement of the wrist assembly 4500 and movement of the tool member 4462.

The wrist assembly 4500 includes a proximal wrist link 4510, a distal wrist link 4610, and a connector link 4580. The connector link 4580 is rotatably coupled between the distal wrist link 4610 and the proximal wrist link 4510. Specifically, the distal wrist link 4610 is rotatable with reference to the connector link 4580 about a distal connector link rotation axis A2 and the proximal wrist link 4510 is rotatable with reference to the connector link 4580 about a proximal connector link rotation axis A3. This arrangement can produce rotation of the distal wrist link 4610 relative to the proximal wrist link 4510 about a wrist rotation axis A4.

Referring FIGS. 7A-7B, the distal wrist link 4610 includes a proximal end portion and a distal end portion. The distal end portion includes a first tool support 4651 (which can be a clevis ear) and a second tool support 4661 (which can be clevis ear). The second tool support 4661 is opposite the first tool support 4651 and together they define a tool member rotation axis A1. The proximal end portion includes a distal connector link receptacle 4655. The distal link connector receptacle 4655 can be a volume within the distal wrist link (i.e., a volume defined by a side wall of the distal wrist link 4610) within which the distal end 4582 of the connector link 4580 is rotatably coupled. The distal link connector receptacle 4655 and the side wall of the distal wrist link 4610 that defines the distal link connector receptacle 4655 can have any suitable shape, size, or orientation to couple the connector link 4580 therein such that the distal wrist link 4610 is rotatable with reference to the connector link 4580 about the distal connector link rotation axis A2 (arrow GG). For example, in some embodiments, the distal link connector receptacle 4655 can include opposing cylindrical portions that are aligned with (or define) the distal connector link rotation axis A2. The side wall surrounding the cylindrical portions can retain the distal end 4582 of the connector link 4580 in the distal link connector receptacle 4655 while also allowing rotation of distal link 4610 with respect to the connector link 4580. Said another way, the distal connector link receptacle 4655 is configured to resist withdrawal of the connector link 4580) from the distal wrist link 4610. In some embodiments, the distal connector link rotation axis A2 is non-parallel to the tool member rotation axis A1.

The end effector 4460 includes at least one tool member 4462 that is coupled to rotate between the first tool support 4651 and the second tool support 4661 about the tool member rotation axis A1. More particularly, the tool member 4462 includes a contact portion and a pulley portion 4467. The contact portion is configured to engage or manipulate a target tissue during a surgical procedure. For example, in some embodiments, the contact portion can include an engagement surface that functions as a gripper, cutter, tissue manipulator, or the like. In other embodiments, the contact portion can be an energized portion of the tool member that is used for cauterization or electrosurgical procedures. The pulley portion 4467 is rotatably coupled to the first tool support tool support 4651 and the second tool support 4661 by a pin 4670. The pulley portion 4467 includes a contact surface or other structure to which an actuator or tension element (not shown) can be coupled. In this manner, when actuated the tool member 4462 can rotate about the tool member rotation axis A1 in a direction of arrows FF. Although only one tool member 4462 is shown, in other embodiments, the medical device 4400 can include two or more moving tool members that cooperatively perform gripping or shearing functions. Thus, the tool member rotation axis A1 can also function as a cutting axis as tool members rotate in opposition to each other as described herein.

Referring to FIG. 7D, the connector link 4580 includes a proximal end portion 4581 and a distal end portion 4582 and defines a longitudinal axis AL that extends between the proximal end portion 4581 and the distal end portion 4582. The distal end portion 4582 includes a first protrusion 4585 and a second protrusion 4586 that are coupled within the distal link connector receptacle 4655 of the wrist link 4610. As described above, the distal link connector receptacle 4655 can retain the distal end 4582 of the connector link 4580 while also allowing rotation of distal link 4610 with respect to the connector link 4580. The proximal end portion 4581 includes a first protrusion 4583 and a second protrusion 4584 that are coupled within the proximal link connector receptacle 4555 of the wrist link 4510, described in more detail below.

The proximal wrist link 4510 includes a proximal end portion and a distal end portion. The proximal end portion is coupled to a shaft, spacer, or other structure (not shown) for coupling the wrist assembly 4500 to a mechanical structure (e.g., similar to the mechanical structure 6700) for actuation of the device. The distal end portion includes a proximal connector link receptacle 4555. The proximal link connector receptacle 4555 can be a volume within the proximal wrist link (i.e., a volume defined by a side wall of the proximal wrist link 4510) within which the proximal end 4581 of the connector link 4580 is rotatably coupled. The proximal link connector receptacle 4555 and the side wall of the proximal wrist link 4510 that defines the proximal link connector receptacle 4555 can have any suitable shape, size, or orientation to couple the connector link 4580 therein such that the proximal wrist link 4510 is rotatable with reference to the connector link 4580 about the proximal connector link rotation axis A3 (arrow HH). For example, in some embodiments, the proximal link connector receptacle 4555 can include opposing cylindrical portions that are aligned with (or define) the proximal connector link rotation axis A3. The opposing cylindrical portions are shown in FIG. 7E as the portion 4557.

Either (or both) of the proximal link connector receptacle 4555 or the distal link connector receptacle 4655 is configured to accept insertion of the connector link 4580 at a first orientation of the connector link 4580 about the longitudinal axis L_{A} of the connector link. Either (or both) the proximal link connector receptacle 4555 or the distal link connector receptacle 4655 is also configured to resist withdrawal of the connector link at a second orientation of the connector link 4580 about the longitudinal axis L_{A}. Similarly stated, either (or both) of the proximal wrist link 4510 or the distal wrist link 4610 are structured so that the end of the connector link 4580 can be inserted into the link connector receptacle while also remaining captive (or locked) within the link connector receptacle. In this manner, the connector link 4580 can be coupled to either wrist link (i.e., within the proximal link connector receptacle 4555 or the distal link connector receptacle 4655) in an efficient manner. For example, as shown in FIG. 7E, in some embodiments, an end surface of the proximal wrist link 4510 defines an insertion opening 4556 into the proximal connector link receptacle 4555. A shape of the insertion opening 4556 corresponds with a shape of the proximal end portion 4581 of the connector link 4580 to allow the proximal end portion 4581 to pass therethrough during assembly. More specifically, the shape of the insertion opening 4556 (when viewed in a plane normal to the longitudinal axis L₇₉ as shown in FIG. 7E) defines an insertion major axis Aᵢₙₛ. The insertion major axis Aᵢₙₛ represents a line that passes through the furthest points on the cross-sectional shape of the insertion opening 4556. To pass through the insertion opening 4556, the connector link 4580 must be rotated as shown by the arrow JJ to the first orientation (shown by the angle Θ in FIG. 7E). In this manner the proximal end portion 4581 of the connector link 4580 is aligned with the insertion opening 4556.

After the proximal end portion 4581 of the connector link 4580 is moved through the insertion opening 4556 and into the proximal link connector receptacle 4555, the connector link 4580 is rotated towards the second orientation, as shown by the arrow JJ. When the connector link is in the second orientation, the first protrusion 4583 and the second protrusion 4584 are retained within the proximal link connector receptacle 4555 and are aligned with the proximal connector link rotation axis A3. Although proximal wrist link 4510 is shown as including the insertion opening 4556, in other embodiments, the distal wrist link 4610 can include a similar insertion opening.

In addition to being coupled together via the connector link 4580, in some embodiments, the distal wrist link 4610 is in rolling contact with the proximal wrist link 4510 as the distal wrist link 4610 rotates with reference to the proximal wrist link 4510.

In some embodiments, either (or both) of the distal wrist link 4610 or proximal wrist link 4510 can be constructed from multiple discrete link pieces, similar to the wrist links 2610 and 3610 described above. In other embodiments, either (or both) of the distal wrist link 4610 or the proximal wrist link 4510 can be constructed monolithically (i.e., as a single structure).

In some embodiments, the medical device 4400 can include one or more tension elements that are coupled to a mechanical structure, and movement of the tension elements can produce the desired movement of the wrist assembly 4500, the tool member 4462, or both. Thus, although neither of the distal wrist link 4610 nor the proximal wrist link 4510 are shown as defining a channel that forms a tension element guide channel, in some embodiments, either (or both of) the distal wrist link 4610 or the proximal wrist link 4510 can define one or more tension element guide channels (not shown). Any tension element guide channels can have any suitable size, shape, or contour to provide a desired path for a tension element to pass therethrough, such as the shape of the tension element guide channels 6515, 6615 described herein.

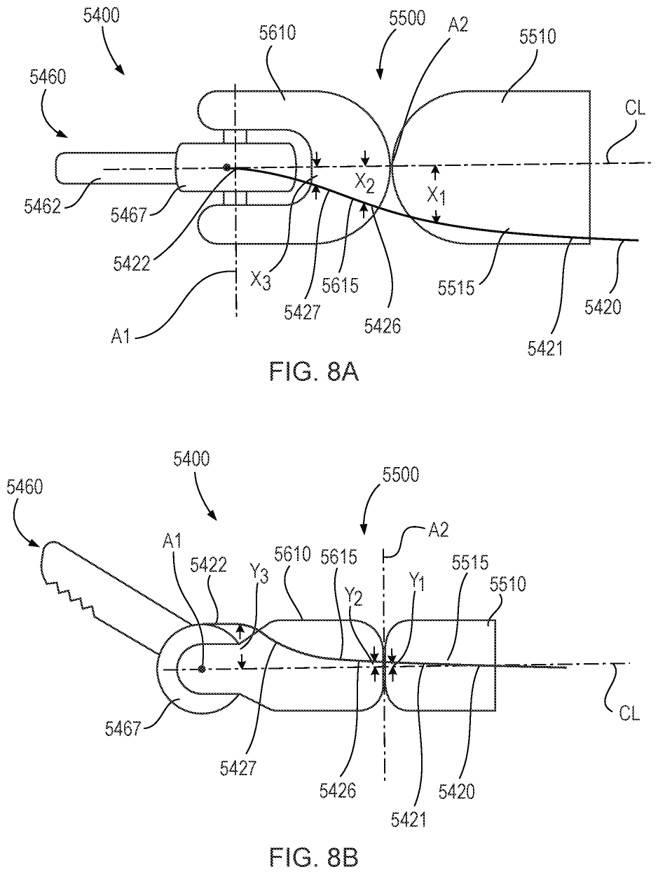

As described above, any of the wrist assemblies described herein can include any suitable tension element guide channels with a desired size, shape, or contour to provide a desired path for the tension element to pass therethrough. For example, FIGS. 8A and 8B are schematic illustrations of a portion of a medical device 5400 according to an embodiment. In some embodiments, the medical device 5400 or any of the components therein are optionally parts of an instrument of a surgical system that performs surgical procedures, and which can include a manipulator unit, a series of kinematic linkages, a series of cannulas, or the like. The medical device 5400 (and any of the instruments described herein) can be used in any suitable surgical system, such as the MIRS system 1000 shown and described above. The medical device 5400 includes a wrist assembly 5500, an end effector 5460, and a tension element 5420. Although only one tool member 5462 is shown, one or more additional tool members can be included.

The wrist assembly 5500 includes a proximal wrist link 5510 and a distal wrist link 5610 that is rotatably coupled to the proximal wrist link about a wrist rotation axis A2. The wrist assembly 5500 defines a longitudinal center line CL. Although the longitudinal center line CL is shown as being linear, when the wrist assembly is moved into different orientations (i.e., when the second link 5610 rotates relative to the first link 5510), the longitudinal center line CL can be curved.

The proximal wrist link 5510 defines a first tension element guide channel 5515 and the distal wrist link 5610 defines a second tension element guide channel 5615. The tension element guide channels 5515, 5615 are shaped and contoured to produce the desired path for the tension elements (e.g., tension element 5420 to pass therethrough). Considerations informing the shape of the first tension element guide channel 5515 and the second tension element guide channel 5615 (and any tension element guide channels described herein) include reducing friction losses (from tension element movement within the channels), maintaining the tension elements within a distal boundary of the wrist assembly 5500, minimizing a fleet angle of the tension elements with respect to their connection to the end effector 5460, and positioning the tension elements relative to the longitudinal center line CL to maximize the torque that can be applied about one of the axes of rotation of the wrist assembly 5500 or end effector 5460. For example, the tension element guide channel 5515 can be shaped to reduce sharp bends, which can reduce friction losses when the tension element 5420 is moved therein.

The end effector 5460 includes at least one tool member 5462 that is coupled to rotate at the distal end portion of the distal wrist link 5610 about the tool member rotation axis A1. More particularly, the tool member 5462 includes a contact portion and a pulley portion 5467. The contact portion is configured to engage or manipulate a target tissue during a surgical procedure. The pulley portion 5467 is rotatably coupled to the distal wrist link 5610 and includes a contact surface or other structure to which an actuator or tension element (not shown) can be coupled. In this manner, when actuated the tool member 5462 can rotate about the tool member rotation axis A1.

The shape of the guide channels can be described with reference to multiple different planes that are normal to the longitudinal center line CL of the wrist assembly 5500. The planes can be referred to as X-Y planes and are defined by a first dimension (identified as an X dimension in FIG. 8A) and a second dimension (identified as a Y dimension in FIG. 8B). The Y dimension is parallel to the second rotation axis A2 (i.e., the pitch axis), and the X dimension is parallel to the first rotation axis A1 (i.e., the yaw axis). The proximal portion 5421 of the tension element is routed within the first tension element guide channel 5515, which is shaped and sized so that the first proximal end 5421 of the tension element 5420 is offset from the center line CL by a first X distance X₁ and a first Y distance Y₁. Because the Y-axis is parallel to the second rotation axis A2 (i.e., the pitch axis), it is desirable to have a large value for the first distance X₁ to maximize the torque that can be applied by the tension elements when rotating the second link 5610 about the second rotation axis A2. Additionally, because the tension elements must be routed within a distal boundary, increasing the first distance X₁ will result in a smaller value for the first distance Y₁.

The tension element 5420 includes a first central portion 5426 and a second central portion 5427. The first central portion 5426 of the tension element 5420 is between the first proximal portion 5421 and the distal portion 5422, and is the length of tension element that is between the exit of the tension element guide channel 5515 of the first link 5510 and within the entry point of the tension element guide channel 5615 of the second link 5610. Thus, the first central portion 5426 of the tension element 5420 is within the tension element guide channel 5615 at a second plane. The second central portion 5427 of the tension element 5420 is between the first proximal portion 5421 and the distal portion 5422, and is the length of tension element that exits the tension element guide channel 5615 and is coupled to the tool member. Thus, the second central portion 5427 of the tension element 5420 is within the tension element guide channel 5615 at a third plane. As shown in FIGS. 8A and 8B, the tension element guide channel 5615 is curved in both an X dimension and a Y dimension to produce the desired performance.

Specifically, the first tension element guide channel 5615 is shaped and sized so that the first central portion 5426 of the tension element 5420 is offset from the center line CL by a second X distance X₂ and a second Y distance Y₂. Because the Y-axis is parallel to the second rotation axis A2 (i.e., the pitch axis), it is desirable to have a large value for the second distance X₂ to maximize the torque that can be applied by the tension elements when rotating the second link 5610 about the second rotation axis A2. In some embodiments, the second distance X₂ is the same as the first distance X₁. Similarly stated, in some embodiments, the shape and position with respect to the center line CL of the entry portion of the tension element guide channel 5615 (i.e., the portion at the second plane) is the same as the shape and position with respect to the center line CL of the entry portion of the tension element guide channel 5515 (i.e., the portion at the first plane).

As shown, the tension element guide channel 5615 is curved and sized such that the second central portion 5427 of the tension element 5420 is offset from the center line by a third X distance X₃ and a third Y distance Y₃. Because the X-axis is parallel to the first rotation axis A1 (i.e., the yaw axis), it is desirable to have a large value for the third distance Y₃ to maximize the torque that can be applied by the tension elements when rotating the tool member 5462 about the first rotation axis A1. Additionally, because the tension elements must be routed within the distal boundary, increasing the third distance Y₃ will result in a smaller value for the third distance X₃. Here, the third distance Y₃ is greater than the second distance Y₂ and the third distance X₃ is less than the second distance X₂. The smaller third distance X₃ (which brings the tension elements more inboard in the X dimension also reduces the fleet angle between the pulley portion 5467 and the second central portion 5427 of the cable.

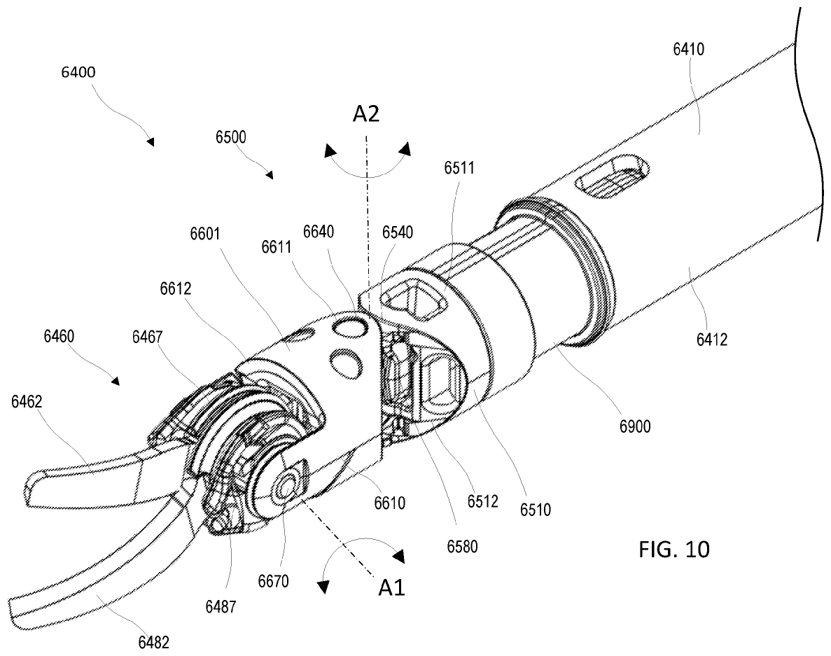

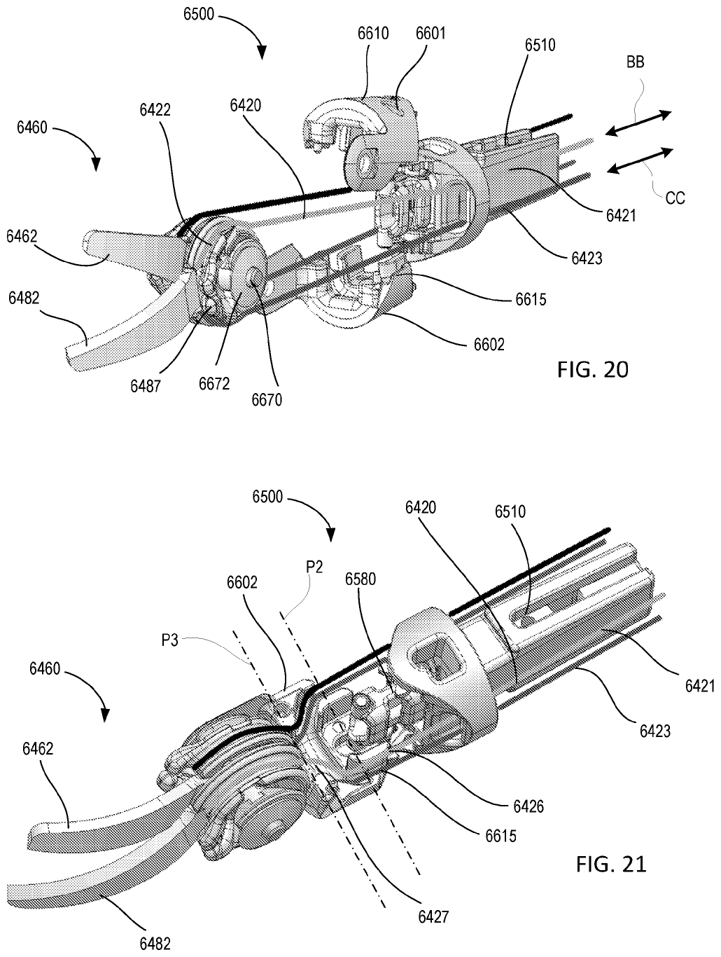

FIGS. 9-23 are various views of an instrument 6400, according to an embodiment. In some embodiments, the instrument 6400 or any of the components therein are optionally parts of a surgical system that performs surgical procedures, and which can include a manipulator unit, a series of kinematic linkages, a series of cannulas, or the like. The instrument 6400 (and any of the instruments described herein) can be used in any suitable surgical system, such as the MIRS system 1000 shown and described above. As shown in FIG. 9, the instrument 6400 defines (or is included within) a distal boundary (or footprint) 6599 that corresponds to a cannula size or other size dictated by the surgical environment. The distal boundary 6599 can be a cylindrical shape having any suitable nominal diameter (e.g., 8 mm, 5 mm or any size therebetween). The instrument 6400 includes a proximal mechanical structure 6700, a shaft 6410 (see, e.g., FIGS. 9 and 10), a distal wrist assembly 6500, a distal end effector 6460, and a set of cables (see FIGS. 20-23 identifying one of the cables as cable 6420). The cables function as tension elements that couple the proximal mechanical structure 6700 to the distal wrist assembly 6500 and end effector 6460. In some embodiments, the cables can be constructed from a polymer as described above for the cable 2420. The instrument 6400 is configured such that movement of one or more of the cables produces rotation of the end effector 6460 about a first axis of rotation A1 (see FIG. 10, which functions as a yaw axis, the term yaw is arbitrary), rotation of the wrist assembly 6500 about a second axis of rotation A2 (see FIG. 10, which functions as a pitch axis), a cutting rotation of the tool members of the end effector 6460 about the first axis of rotation A1, or any combination of these movements. Changing the pitch or yaw of the instrument 6400 can be performed by manipulating the cables in a similar manner as that described with reference to the device 2400 described in copending U.S. Provisional Patent Application Ser. No. 63/233,904, entitled “Surgical Instrument Cable Control and Routing Structures,” the disclosure of which is incorporated herein by reference in its entirety.

The proximal mechanical structure 6700 produces movement of each cable (including the cable 6420) to produce the desired movement (pitch, yaw, or grip) at the wrist assembly 6500 and the end effector 6460. Specifically, the proximal mechanical structure 6700 includes components and controls to move some of the cables in a proximal direction (i.e., to pull in certain tension members) while simultaneously allowing the distal movement (i.e., releasing or “paying out”) of other of the cables. In this manner, the proximal mechanical structure 6700 can cause the desired movement while also maintaining the desired tension within the cables. As shown in FIG. 9, the proximal mechanical structure 6700 includes a set of capstans that rotate or “wind” a proximal portion of any of the cables to produce the desired cable movement. In some embodiments, the two proximal ends of the cable 6420 (see e.g., the first proximal end portion 6421 and the second proximal end portion 6423 shown in FIGS. 20 and 21), which are associated with opposing directions of a single degree of freedom, are connected to two independent drive capstans 6710 and 6720. This arrangement, which is generally referred to as an antagonist drive system, allows for independent control of the movement of (e.g., pulling in or paying out) each of the ends of the cable 6420. The mechanical structure 6700 produces movement of the cable 6420, which operates to produce the desired articulation movements (pitch, yaw, or grip) at the wrist assembly 6500 and end effector 6460. Accordingly, the mechanical structure 6700 includes components and controls to move the first proximal end portion 6421 of the cable 6420 via the first capstan 6710 in a first direction (e.g., a proximal direction) and to move the second proximal end portion 6423 of the cable 6420 via the second capstan 6720 in a second opposite direction (e.g., a distal direction). The mechanical structure 6700 can also move both proximal end portions of the cable 6420 in the same direction. In this manner, the mechanical structure 6700 can maintain the desired tension within the cables.

Referring to FIG. 20, in use, the first capstan 6710 can be actuated to move the first proximal portion 6421 of the cable 6420 in a first direction along arrows BB. Simultaneously, the second capstan 6720 can be actuated to move the second proximal end portion 6723 of the cable 6420 in an opposite direction as the first proximal portion 2423 along arrows CC. Thus, the opposite movement of the first proximal portion 6421 and the second proximal portion 6423 causes the end effector 6460 to rotate (via the cable 6420 connection to the end effector 6460) about the rotational axis A1 (see FIG. 10; e.g., yaw movement). In a similar way, accurate rotation in pitch around a second axis A2 (see FIG. 10; e.g., pitch; orthogonal to the yaw axis A1 described above) can be controlled. Specifically, the first capstan 6710 and the second capstan 6720 can each be actuated to move the first proximal portion 6421 and the second proximal portion 6423 of the cable 2420 together in the same direction (along arrows BB and CC, respectively). This causes the wrist assembly 6500 to rotate (via the cable 6420 connection through the wrist assembly 6500 and to the end effector 6460) about a second rotation axis A2 in the direction (e.g., pitch movement).

In some embodiments, the mechanical structure 6700 can include any of the assemblies or components described in U.S. Provisional Patent Application Ser. No. 63/233,904, entitled “Surgical Instrument Cable Control and Routing Structures,” the disclosure of which is incorporated herein by reference in its entirety. In other embodiments, however, any of the medical devices described herein can have the two ends of the cable wrapped about a single capstan. This alternative arrangement, which is generally referred to as a self-antagonist drive system, operates the two ends of the cable using a single drive motor.

Moreover, although the mechanical structure 6700 is shown as including capstans, in other embodiments, a mechanical structure can include one or more linear actuators that produce translation (linear motion) of a portion of the cables. Such proximal mechanical structures can include, for example, a gimbal, a lever, or any other suitable mechanism to directly pull (or release) an end portion of any of the cables. For example, in some embodiments, the proximal mechanical structure 6700 can include any of the proximal mechanical structures or components described in U.S. Patent Application Pub. No. US 2015/0047454 A1 (filed August 15, 2014), entitled “Lever Actuated Gimbal Plate,” or U.S. Pat. No. 6,817,974 B2 (filed June 28, 2001), entitled “Surgical Tool Having Positively Positionable Tendon-Actuated Multi-Disk Wrist Joint,” each of which is incorporated herein by reference in its entirety.

The shaft 6410 can be any suitable elongated shaft that is coupled to the wrist assembly 6500 and to the mechanical structure 6700. Specifically, the shaft 6410 includes a proximal end 6411 that is coupled to the mechanical structure 6700, and a distal end 6412 that is coupled to the wrist assembly 6500 (e.g., a proximal link of the wrist assembly 6500). The instrument shaft 6410 defines a passageway or series of passageways through which the cables and other components (e.g., electrical wires, ground wires, or the like) can be routed from the proximal mechanical structure 6700 to the wrist assembly 6500. In some embodiments, the shaft 6410 can be formed, at least in part with, for example, an electrically conductive material such as stainless steel. In such embodiments, the shaft may include any of an inner insulative cover or an outer insulative cover. Thus, the shaft 6410 can be a shaft assembly that includes multiple different components. For example, as shown in FIGS. 9 and 10, the shaft 6410 can include (or be coupled to) a spacer 6900 that provides the desired fluid seals, electrical isolation features, and any other desired components for coupling the wrist assembly 6500 to the shaft 6410. Similarly stated, although the wrist assembly 6500 (and other wrist assemblies or links described herein) are described as being coupled to the shaft 6410, it is understood that any of the wrist assemblies or links described herein can be coupled to the shaft via any suitable intermediate structure, such as a spacer 6900 and a cable guide, or the like. In some embodiments, any of the medical devices described herein can include any of the spacers, seals, cable guides, or other structure as described in U.S. Provisional Patent Application Ser. No. Attorney Docket No. P06585-US-PRV, filed on the same date herewith and entitled “Surgical Instrument Including Electrical and Fluid Isolation Features,” the disclosure of which is incorporated herein by reference in its entirety.

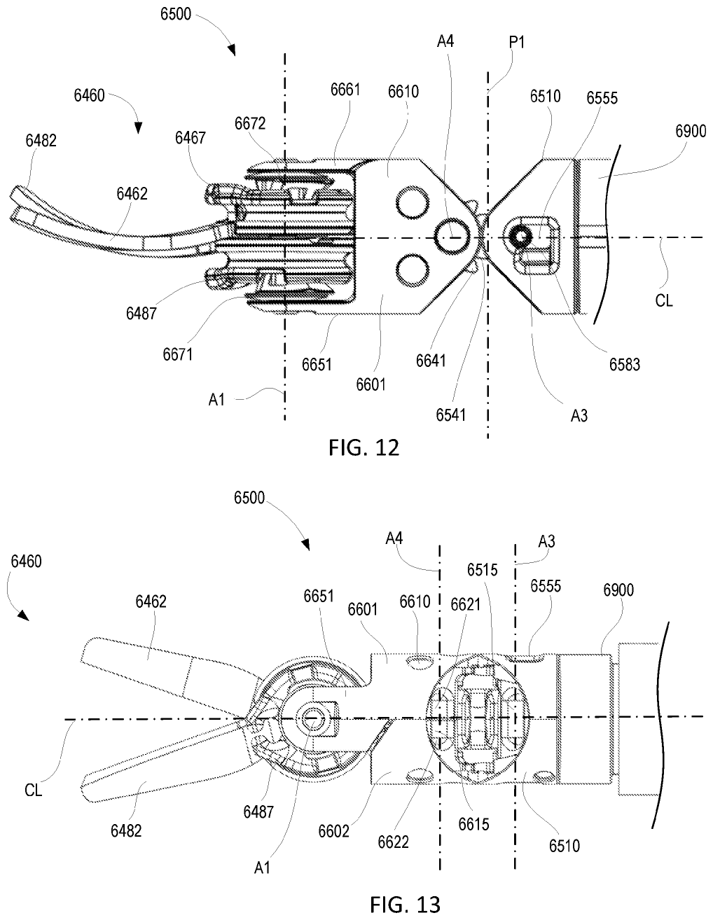

Referring to FIG. 10, the wrist assembly 6500 (also referred to as a joint assembly) includes a first link 6510 (which functions as a proximal wrist link), a second link 6610 (which functions as a distal wrist link) and a connector link 6580. As described in detail herein, the connector link 6580 is coupled between the first link 6510 and the second link 6610 to form the articulating wrist assembly 6500. The first link 6510 is coupled to the connector link 6580 via a pinned joint such that the first link 6510 is rotatable with reference to the connector link 6580 about a third rotation axis A3 (which functions as a proximal connector link rotation axis; see FIGS. 12 and 13). The second link 6610 is also coupled to the third link 6580 via a pinned joint such that the second link 6610 is rotatable with reference to the connector link 6580 about a fourth rotation axis A4 (which functions as a distal connector link rotation axis; see FIGS. 12 and 13). In this manner, the connector link 6580 maintains the coupling between the first link 6510 and the second link 6610 during rotation of the second link 6610 relative to the first link 6510. Further, as described below, the distal end portion 6512 of the first link 6510 includes a joint portion 6540 that is rotatably coupled to a mating joint portion 6640 at the proximal end portion 6611 of the second link 6610. In this manner, the second link 6610 rotates relative to the first link 6510 about the second rotation axis A2 (which functions as the pitch axis, the term pitch is arbitrary). Because the joint between the first link 6510 and the second link 6610 is not a pinned joint, the second rotation axis A2 will move relative to the first link 6510 during rotation of the second link 6610.

The wrist assembly 6500 defines a longitudinal center line CL (see FIGS. 12, 13, 15, 22, and 23). Although the longitudinal center line CL is shown as being linear, when the wrist assembly is moved into different orientations (i.e., when the second link 6610 rotates relative to the first link 6510), the longitudinal center line CL can be curved. The distal boundary 6599 can be a cylindrical boundary with the center being along the longitudinal center line CL.

The first link 6510 has a proximal portion 6511 and a distal portion 6512. The proximal portion 6511 includes a coupling protrusion that is coupled to the spacer 6900. The proximal portion 6511 can be coupled to the spacer 6900 via any suitable mechanism. For example, in some embodiments, the proximal portion 6511 can be matingly disposed within a portion of the spacer 6900 (e.g., via an interference fit). In some embodiments, the proximal portion 6511 can include one or more protrusions, recesses, openings, or connectors that couple the proximal portion 6511 to the spacer 6900. In some embodiments, some portions of the wrist assembly 6500 are formed with a metallic material and are used in the delivery of electrical energy to the tool members 6462 and 6482. For example, the first link 6510 and the second link 6610 can be formed with a metallic material. Accordingly, as shown, the coupling protrusion of the proximal portion 6511 includes an interior region that forms an electrical connector 6573 configured to receive an electrical wire (not shown) to electrically couple the electrical wire to the first link 6511. The electrical connector 6573 can be a push-in type connector that includes sharp protrusions configured to strip away portions of the insulation from the electrical wire to establish electrical coupling between the wire and the first link 6510.

The distal portion 6512 of the first link 6510 includes a joint portion 6540 that is rotatably coupled to a mating joint portion 6640 of the second link 6610. Specifically, the joint portion 6540 includes a first set of teeth 6541, a second set of teeth 6542 and curved contact surfaces 6544. The first set of teeth 6541 intermesh with the corresponding first set of teeth 6641 on the second link 6610 and the second set of teeth 6542 intermesh with the corresponding second set of teeth 6642 on the second link 6610. When the second link 6610 rotates relative to the first link 6510 (i.e., pitch rotation about the second rotation axis A2), the curved contact surface 6544 are in rolling contact with the corresponding curved contact surfaces 6644 of the second link 6610. The mating joint portions 6540 and 6640 can be similar to those shown and described in U.S. Patent Application Pub. No. US 2017/0120457 A1 (filed Feb. 20, 2015), entitled “Mechanical Wrist Joints with Enhanced Range of Motion, and Related Devices and Methods,” which is incorporated herein by reference in its entirety.

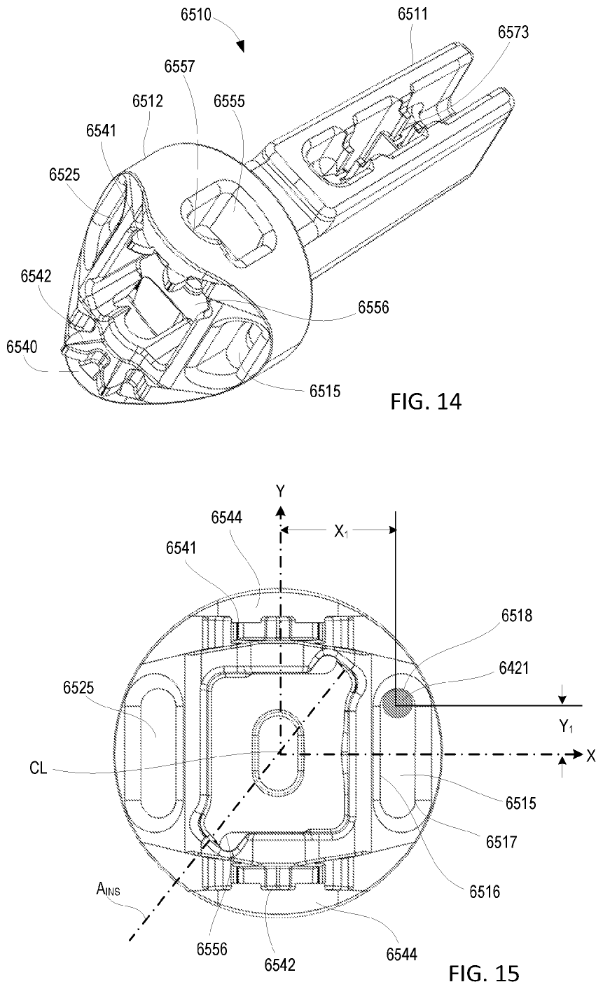

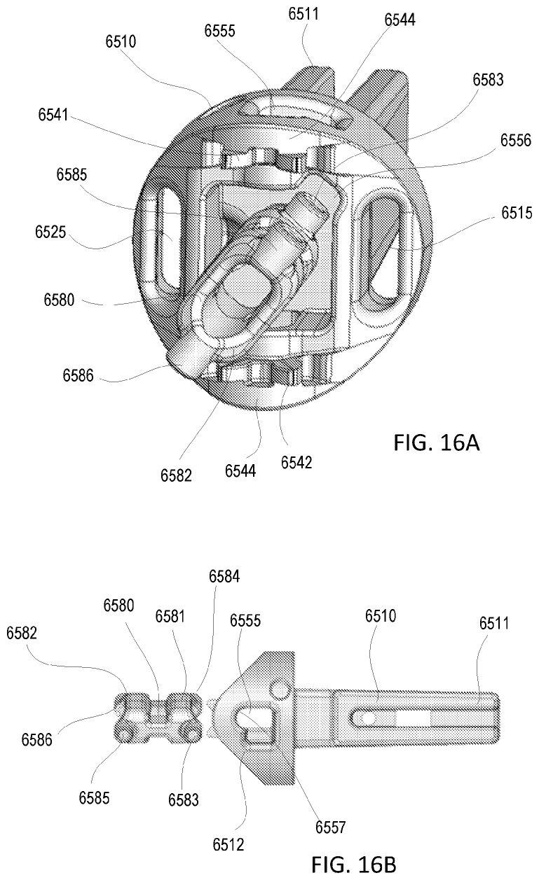

The first link 6510 includes a proximal connector link receptacle 6555 that receives and retains a proximal end 6581 of the connector link. Similarly stated, a side wall of the first link 6510 defines a volume within the first link 6510 that functions as the proximal connector link receptacle 6555. As shown in FIGS. 14 and 16B, a side wall portion 6557 of the first link 6510 defines opposing cylindrical portions that receive the cylindrical protrusions 6583, 6584 of the proximal end 6581 of the connector link 6580. The opposing cylindrical portions are aligned with (or define) the proximal connector link rotation axis A3. In this manner, the proximal end 6581 of the connector link 6580 is rotatably coupled within the first link 6510 about the third rotation axis A3 via the proximal connector link receptacle 6555.

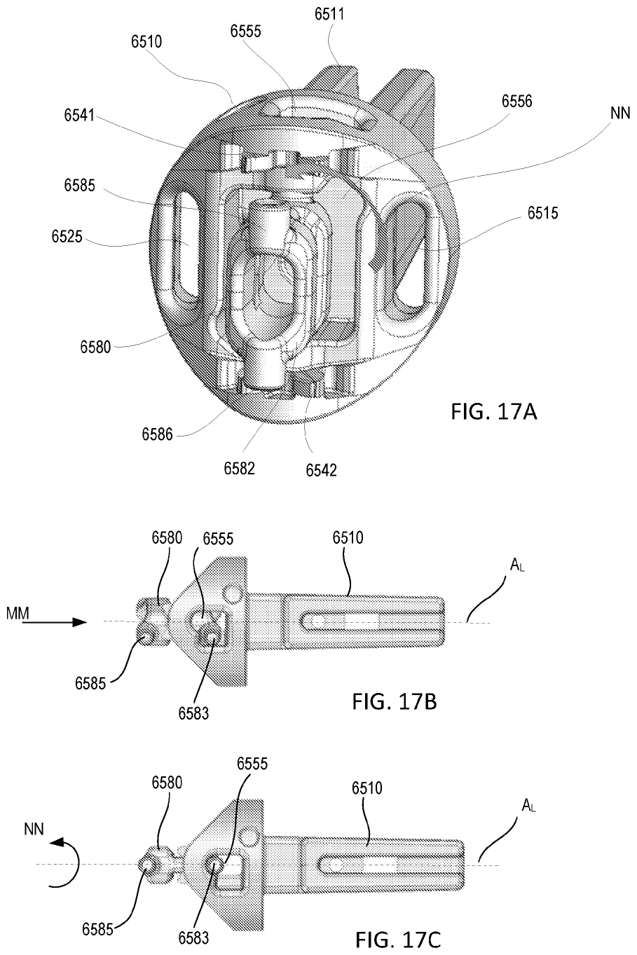

As shown in FIGS. 16A-17C, the proximal link connector receptacle 6555 is configured to accept insertion of the connector link 6580 when then connector link is at a first orientation about the longitudinal axis L_{A} of the connector link 6580 (see FIGS. 16A, 16B and 17B). Similarly stated, the proximal wrist link 6510 is structured so that the proximal end 6581 of the connector link 6580 can be inserted into the link connector receptacle 6555 while also remaining captive (or locked) therein.

For example, as shown in FIGS. 16A and 17A, an end surface of the first link 6510 defines an insertion opening 6556 into the proximal connector link receptacle 6555. The shape of the insertion opening 6556 corresponds with the shape of the proximal end portion 6581 of the connector link 6580 to allow the proximal end portion 6581 to pass therethrough during assembly. More specifically, the shape of the insertion opening 6556 (when viewed in a plane normal to the longitudinal axis L_{A} as shown in FIG. 15) defines an insertion major axis Aᵢₙₛ. The insertion major axis Aᵢₙₛ represents a line that passes through the furthest points on the cross-sectional shape of the insertion opening 6556. To pass through the insertion opening 6556, the connector link 6580 must be rotated to the first orientation (FIGS. 16A, 16B and 17B). In this manner the proximal end portion 6581 of the connector link 6580 is aligned with the insertion opening 6556. The connector link 6580 is then inserted through the insertion opening 6555 as shown by the arrow MM in FIG. 17B.

After the proximal end portion 6581 of the connector link 6580 is moved through the insertion opening 6556 and into the proximal link connector receptacle 6555, the connector link 6580 is rotated towards the second orientation, as shown by the arrow NN in FIGS. 17A and 17C. When the connector link is in the second orientation, the first protrusion 6583 and the second protrusion 6584 are retained within the proximal link connector receptacle 6555 and are aligned with the third rotation axis A3. Thus, the proximal link connector receptacle 6555 is also configured to resist withdrawal of the connector link 6580 when the connector link 6580 is at the second orientation about the longitudinal axis L_{A}. Although first link 6510 is shown as including the insertion opening 6556, in other embodiments, any of the wrist links herein can include a similar insertion opening.

Referring to FIGS. 14 and 15, the first link defines a first cable guide channel 6515 and a second cable guide channel 6525. Although details of the cable guide channels are discussed below with reference to the first cable guide channel 6515 and the cable 6420, it should be understood that the structure and function disclosed also applies to the second cable guide channel 6525 and any of the cable guide channels and cables described herein. The cable guide channels are shaped and contoured to produce the desired path for the cables (e.g., cable 6420 to pass therethrough). Considerations informing the shape of the first cable guide channel 6515 and the second cable guide channel 6525 (and any cable guide channels described herein) include reducing friction losses (from cable movement within the channels), maintaining the cables within the distal boundary 6599, minimizing a fleet angle of the cables with respect to their connection to the end effector 6460, and positioning the cables relative to the longitudinal center line CL to maximize the torque that can be applied about one of the axes of rotation of the wrist assembly 6500 or end effector 6460. For example, the cable guide channel 6515 can be shaped to reduce sharp bends, which can reduce friction losses when the cable 6420 is moved therein.

As shown, the wrist assembly 6500 does not include (i.e., is devoid of) any pulleys or rollers within the cable guide channel 6515 and the cable guide channel 6615. Similarly stated, no portion of the cable 6420 (including the proximal portion 6421 within the first link 6510, the first central portion 6426, or the second central portion 6427 within the second link 6610) contacts a pulley or roller. Rather, the side walls of the first link and the second link that define the cable guide channel 6515 and the cable guide channel 6615 are shaped and contoured to provide the desired friction and bending characteristics for routing the cables therethrough. Thus, the wrist assembly 6500 can be referred to as a pulley-less wrist assembly. Additionally, as shown in FIGS. 20 and 21, there is no central structure or guide surface within the wrist assembly that is between the first proximal portion 6421 and the second proximal portion 6423 as the cable passes through the wrist assembly 6500.

FIG. 15 shows a side view of the first link within plane P1 (as identified in FIG. 12). Plane P1 is normal to the longitudinal center line CL of the wrist assembly 6500. The plane P1 is defined by a first dimension (identified as the X-axis in FIG. 15) and a second dimension (identified as the Y-axis in FIG. 15). The Y-axis is parallel to the second rotation axis A2 (i.e., the pitch axis), the third rotation axis A3, and the fourth rotation axis A4. The X-axis is parallel to the first rotation axis A1 (i.e., the yaw axis). As shown, the first link 6510 includes an inboard guide surface 6516, an outboard guide surface 6517, and two end guide surfaces 6518 that each form a portion of the boundary of the first guide channel 6515. The inboard guide surface 6516 is closer to the longitudinal center line CL than is the outboard guide surface 6517. The end guide surfaces 6518 are the opposing curved surfaces that connect the inboard guide surface 6516 and the outboard guide surface 6517 at the top and bottom of the guide channel. The first proximal portion 6421 of the cable is routed within the first cable guide channel 6515 in contact with the end guide surface 6518 (the second proximal portion 6423 is routed in contact with the opposing end guide surface, but is not shown in FIG. 15).

As shown in FIG. 15, the first cable guide channel 6515 is shaped and sized so that the first proximal end 6421 of the cable 6420 is offset from the Y-axis by a first X distance X₁ and offset from the X-axis by first Y distance Y₁. Because the Y-axis is parallel to the second rotation axis A2 (i.e., the pitch axis), it is desirable to have a large value for the first distance X₁ to maximize the torque that can be applied by the cables when rotating the second link 6610 about the second rotation axis A2. Additionally, because the cables must be routed within the distal boundary 6599, increasing the first distance X₁ will result in a smaller value for the first distance Y₁. As described in more detail, the cable guide channels through the second link 6610 are configured to route the cable 6420 to reduce the X distance and maximize the Y distance (to increase the torque that can be applied by the cables when rotating the end effector 6460 about the first rotation axis A1 (the yaw axis).

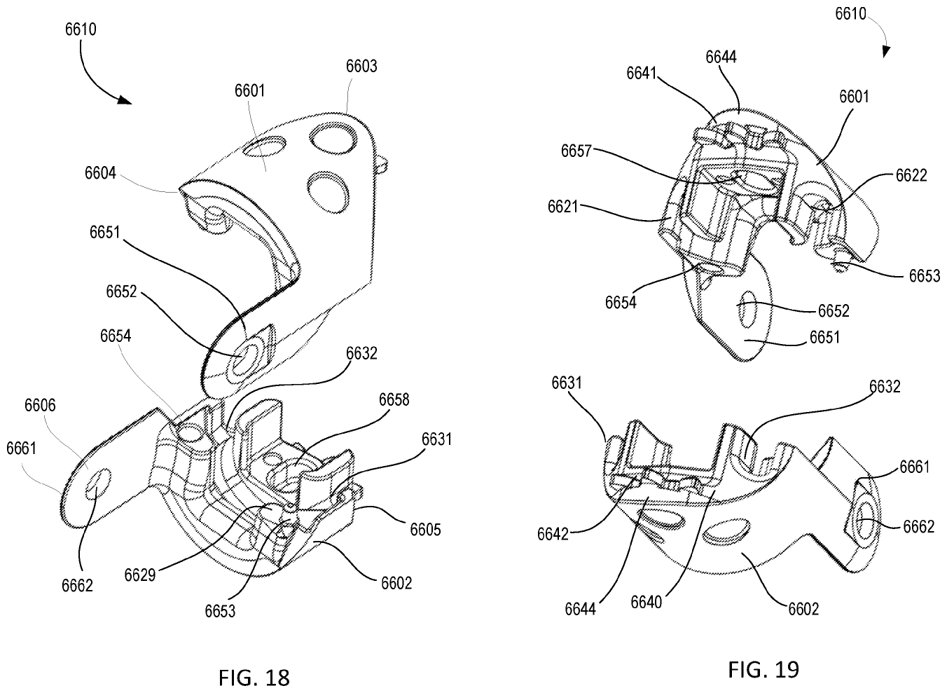

The second link 6610 has a proximal portion 6611 and a distal portion 6612. The proximal portion 6611 is rotatably coupled to the distal portion 6512 of the first link 6510. As described herein, the second link 6610 rotates relative to the first link 6510 about the second rotation axis A2. As described in more detail below, the distal portion 6612 is coupled to the end effector 6460. Referring to FIGS. 18 and 19, the second link 6610 includes a discrete first link piece 6601 and a discrete second link piece 6602. Similarly stated, the first link piece 6601 and the second link piece 6602 are constructed as separate pieces and are later coupled together to form the second link 6610. By forming the second link 6610 from two discrete pieces, the method of assembly of the device 6400 can be made more efficient than that for a device with a monolithically constructed wrist link. For example, as described herein, the cables (e.g., the cable 6420) can be placed into a cable guide channel 6615 before the second link piece 6602 is coupled to the first link piece 6601, thereby eliminating the need to pass loose ends of the cable 6420 through an enclosed channel. The second link piece 6602 can be coupled to the first link piece 6601 by any suitable mechanism (e.g., an adhesive joint, a weld joint, or a mechanical fastener). Moreover, although described separately herein, in some embodiments, the first link piece 6601 and the second link piece 6602 can have the same configuration. Similarly stated, in some embodiments, the first link piece 6601 and the second link piece 6602 can be substantially identical pieces.

The first link piece 6601 includes a proximal end portion 6603 and a distal end portion 6604. The distal end portion 6604 includes a first clevis ear 6651 that defines an opening 6652. The proximal end portion 6603 includes an opening 6657 that, together with the corresponding opening 6658 of the second link piece 6602, form a distal connector link receptacle. The distal connector link receptacle (including the opening 6657 and the opening 6658) receives and retains a distal end 6582 of the connector link 6580. Similarly stated, a side wall of the first link piece 6601 defines the opening 6657 that receives and retains the cylindrical protrusion 6685 of the distal end 6582 of the connector link 6580. The opening 6657 is aligned with the corresponding opening 6658 of the second link piece 6602, and together they define the distal connector link rotation axis A4. The proximal end portion 6603 also includes a first set of teeth 6641 and curved contact surface 6544.

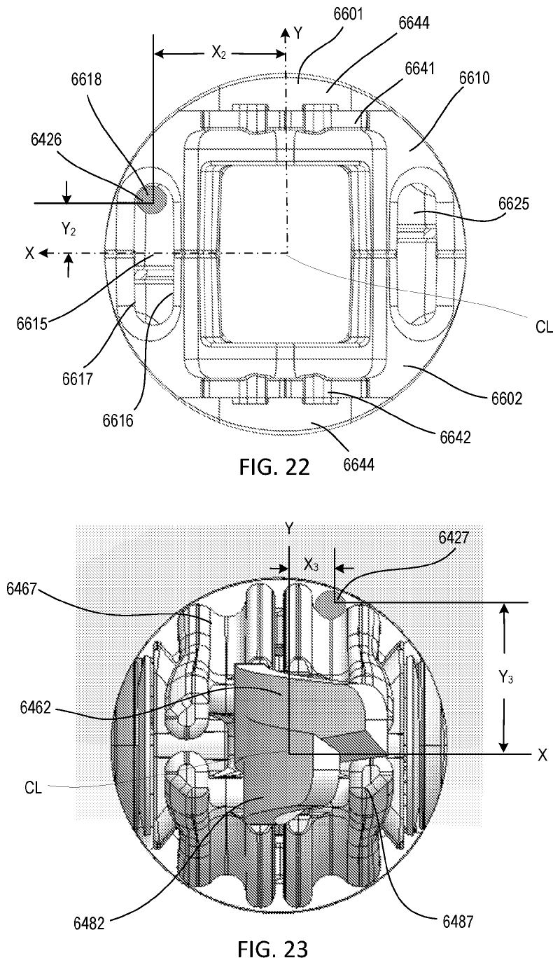

The first link piece 6601 defines a first channel 6621 and a second channel 6622. The first channel 6621 opposes a corresponding first channel 6631 of the second link piece 6602 and the second channel 6622 opposes a corresponding second channel 6632 of the second link piece 6602. In this manner, when the first link piece 6601 is coupled to the second link piece 6602, the first channel 6621 is aligned with the first channel 6631 to define the first cable guide channel 6615 (see FIG. 22). Similarly, the second channel 6622 is aligned with the second channel 6632 to define the second cable guide channel 6625.

The second link piece 6602 includes a proximal end portion 6605 and a distal end portion 6606. The distal end portion 6606 includes a first clevis ear 6661 that defines an opening 6662. The proximal end portion 6605 includes an opening 6658 that, together with the corresponding opening 6657 of the first link piece 6601, form a distal connector link receptacle, as described above. The proximal end portion 6605 also includes a first set of teeth 6642 and curved contact surface 6544. The second link piece 6602 defines the first channel 6631 and the second channel 6632, as described above.

As shown in FIGS. 18 and 19, the first link piece 6601 and the second link piece each include a connection pin 6653 and a connection bore 6654. The connection pin 6653 of the first link piece 6601 is configured to be received within the connection bore 6654 of the second link piece 6602 (see FIG. 18), and vice-versa, to facilitate coupling the first link piece 6601 to the second link piece 6602. When the second link piece 6602 is coupled to the first link piece 6601 to form the second link 6610, the second clevis ear 6661 is opposite the first clevis ear 6651. Thus, the openings 6652 and 6662 are aligned such that a pin 6670 can be coupled between the first clevis ear 6651 and the second clevis ear 6661 to allow rotation of the end effector 6460 about a tool member rotation axis A1. Similarly stated, when the second link piece 6602 is coupled to the first link piece 6601, the second clevis ear 6661 is aligned with the first clevis ear 6651 to define the tool member rotation axis A1. Additionally, when the second link piece 6602 is coupled to the first link piece 6601, the opening 6657 is opposite the opening 6658. Thus, the openings 6657 and 6658 are aligned such that the protrusion 6585 and the protrusion 6586 can be coupled within the openings to allow rotation of the connector link 6580 about the fourth rotation axis A4. The tool member rotation axis A1 is perpendicular to the distal connector link rotation axis A4.

When the second link 6610 is assembled, the end effector 6460 is rotatably coupled to the second link 6610 about the first rotation axis A1. The end effector 6460 includes a first tool member 6462 and a second tool member 6482 that are coupled between the clevis ears 6651 and 6661. The first tool member 6462 includes a contact portion 6464 and a pulley portion 6467. The second tool member 6482 includes a contact portion 6484 and a pulley portion 6487. The pulley portion 6467 is rotatably coupled to the first clevis ear 6651 and the pulley portion 6487 is rotatably coupled to the second clevis ear 6661 by a pin 6670. The pulley portions each include a contact surface or other structure to which cables can be coupled. In this manner, when actuated the tool members 6462, 6482 can rotate about the tool member rotation axis A1 (which functions as the yaw axis; the term yaw is arbitrary).

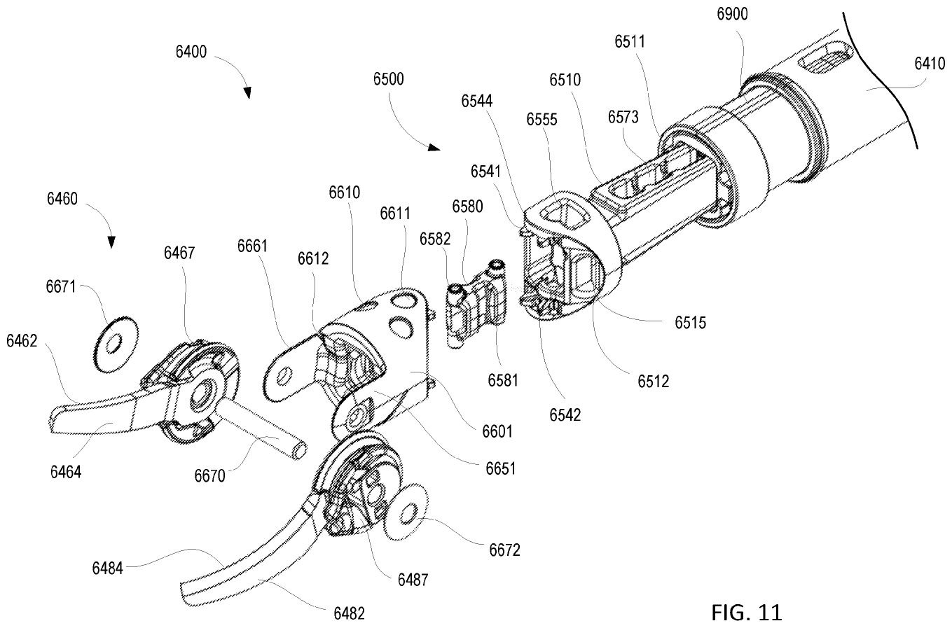

As shown in FIG. 11, a first washer 6671 is between the first clevis ear 6651 and the first tool member 6462 and a second washer 6672 is between the second clevis ear 6661 and the second tool member 6482. The first washer 6671 can be biased to urge the first tool member 6462 away from the clevis ear 6651 and towards the second tool member 6482. The second washer 6672 can be biased to urge the second tool member 6482 away from the clevis ear 6661 and towards the first tool member 6462. Thus, the washers can urge the tool members inboard toward each other to provide the desired interference between the tool member blades, as described in copending U.S. Provisional Patent Application Ser. No. 63/234,662, entitled “Surgical Instrument Shears,” the disclosure of which is incorporated herein by reference in its entirety.

As shown in FIGS. 20-22, when the second link 6610 is assembled, the second link defines a first cable guide channel 6615 and a second cable guide channel 6625. The cable guide channels are shaped and contoured to produce the desired path for the cables (e.g., cable 6420 to pass therethrough). Considerations informing the shape of the first cable guide channel 6615 and the second cable guide channel 6625 (and any cable guide channels described herein) include reducing friction losses (from cable movement within the channels), maintaining the cables within the distal boundary 6599, minimizing a fleet angle of the cables with respect to their connection to the end effector 6460, and positioning the cables relative to the longitudinal center line CL to maximize the torque that can be applied about one of the axes of rotation of the wrist assembly 6500 or end effector 6460.

As one example, as shown in FIG. 18, the second link piece 6601 includes two protrusions 6629 that provide a surface for the connection bore 6654 and the connection pin 6653. The protrusions 6629 also form a curved boundary of each of the cable guide channels 6615, 6625. As shown in FIG. 21, the protrusions 6629 form a part of the outboard surface defining the guide channels and route the cables inboard (i.e., towards the center line CL along the X dimension, as shown in FIG. 23). In this manner, the protrusions 6629 and the guide channels ensure that the second central portion 6627 of the cable exit the second link 6610 in a manner that reduces the fleet angle with respect to the cable connection to the pulley portion 6487.

To further illustrate the cable guide channels within the second link 6610, FIG. 22 shows a side view of the second link within plane P2 (as identified in FIG. 21), which can be characterized as an entry plane (or a plane at the entry point where the cables enter the proximal portion 6611 of the second link 6610). FIG. 23 shows a side view of the second link within plane P3 (as identified in FIG. 21)), which can be characterized as an exit plane (or a plane at the exit point where the cables exit the distal portion 6612 of the second link 6610 and are coupled to the pulley portion 6487) Planes P2 and P3 are normal to the longitudinal center line CL of the wrist assembly 6600. As with plane P1 described above, the planes P2 and P3 are each defined by a first dimension (identified as the X-axis in FIGS. 22 and 23) and a second dimension (identified as the Y-axis in FIGS. 22 and 23). The Y-axis is parallel to the second rotation axis A2 (i.e., the pitch axis), the third rotation axis A3, and the fourth rotation axis A4. The X-axis is parallel to the first rotation axis A1 (i.e., the yaw axis). As shown, the second link 6610 includes an inboard guide surface 6616, an outboard guide surface 6617, and two end guide surfaces 6618 that each form a portion of the boundary of the first guide channel 6615. The inboard guide surface 6616 is closer to the longitudinal center line CL than is the outboard guide surface 6617. The end guide surfaces 6618 are the opposing curved surfaces that connect the inboard guide surface 6616 and the outboard guide surface 6617 at the top and bottom of the guide channel.

The cable 6420 includes a first central portion 6426 and a second central portion 6427. The first central portion 6426 of the cable 6420 is between the first proximal portion 6421 and the distal portion 6422, and is the length of cable that is between the exit of the cable guide channel 6515 of the first link 6510 and within the entry point of the cable guide channel 6615 of the second link 6610. Thus, the first central portion 6426 of the cable 6420 is within the cable guide channel 6615 at the second plane P2. The second central portion 6427 of the cable 6420 is between the first proximal portion 6421 and the distal portion 6422, and is the length of cable that exits the cable guide channel 6615 and is coupled to the tool member. Thus, the second central portion 6427 of the cable 6420 is within the cable guide channel 6615 at the third plane P3. As shown in FIG. 21, the cable guide channel 6615 is curved in both the X dimension and Y dimension to produce the desired performance.

Specifically, as shown in FIG. 22, the first cable guide channel 6615 is shaped and sized so that the first central portion 6426 of the cable 6420 is offset from the Y-axis by a second X distance X₂ and offset from the X-axis by second Y distance Y₂. Because the Y-axis is parallel to the second rotation axis A2 (i.e., the pitch axis), it is desirable to have a large value for the second distance X₂ to maximize the torque that can be applied by the cables when rotating the second link 6610 about the second rotation axis A2. Additionally, because the cables must be routed within the distal boundary 6699, increasing the second distance X₂ will result in a smaller value for the second distance Y₂. In some embodiments, the second distance X₂ is the same as the first distance X₁. Similarly stated, in some embodiments, the shape and position with respect to the center line CL of the entry portion of the cable guide channel 6615 (i.e., the portion at plane P2) is the same as the shape and position with respect to the center line CL of the entry portion of the cable guide channel 6515 (i.e., the portion at plane P1).

As shown in FIG. 23, the cable guide channel 6615 is curved and sized such that the second central portion 6427 of the cable 6420 is offset from the Y-axis by a third X distance X₃ and offset from the X-axis by third Y distance Y₃. Because the X-axis is parallel to the first rotation axis A1 (i.e., the yaw axis), it is desirable to have a large value for the third distance Y₃ to maximize the torque that can be applied by the cables when rotating the tool member 6482 about the first rotation axis A1. Additionally, because the cables must be routed within the distal boundary 6699, increasing the third distance Y₃ will result in a smaller value for the third distance X₃. Here, the third distance Y₃ is greater than the second distance Y₂ and the third distance X₃ is less than the second distance X₂. The smaller third distance X₃ (which brings the cables more inboard in the X dimension also reduces the fleet angle between the pulley portion 6487 and the second central portion 6427 of the cable.

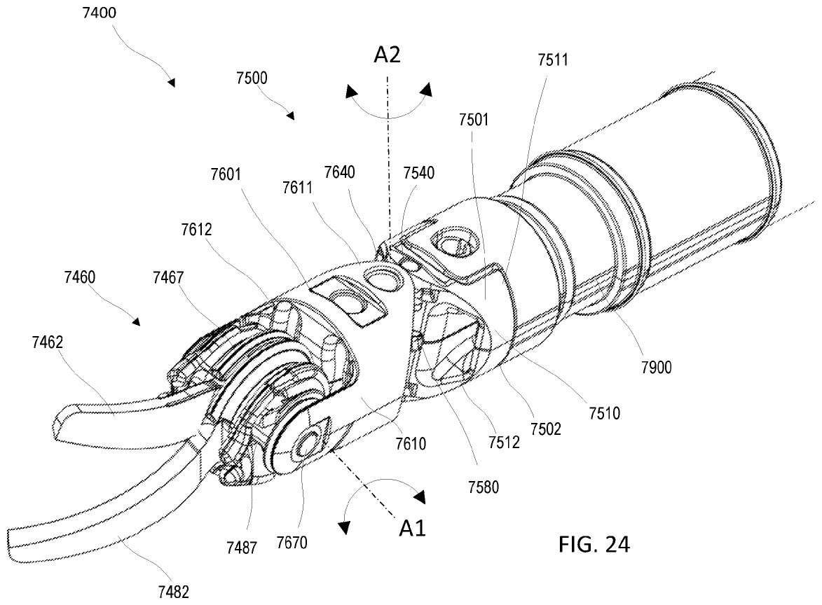

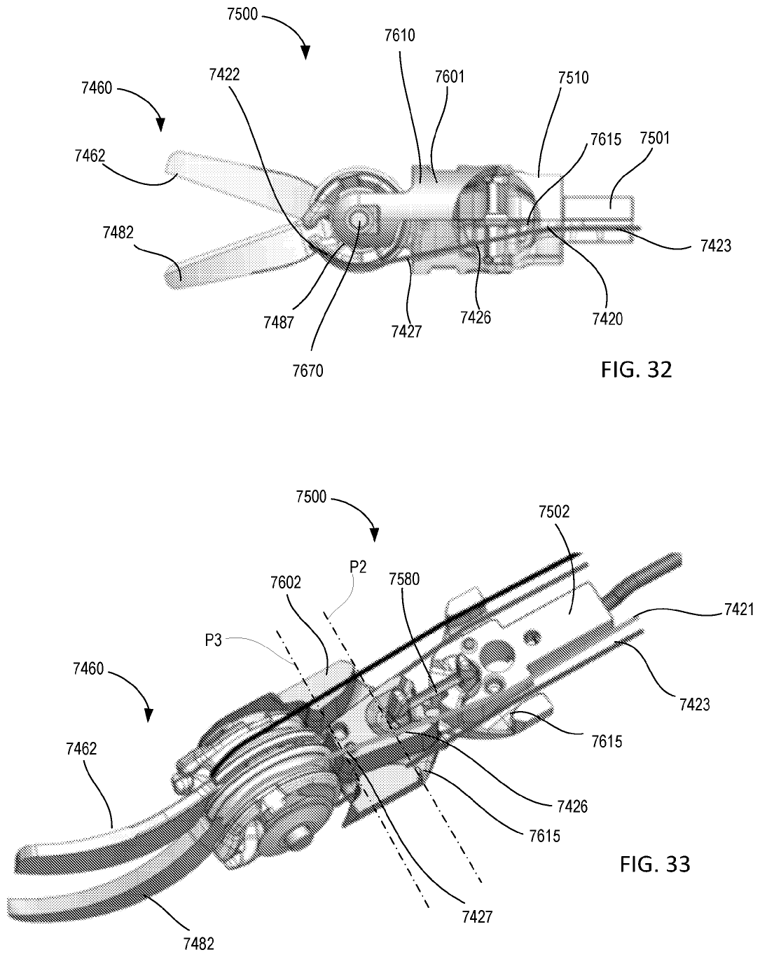

In some embodiments and wrist assembly can include multiple links (e.g., a proximal link and distal link) that are constructed from multiple discrete pieces. For example, FIGS. 24-37 are various views of an instrument 7400, according to an embodiment. In some embodiments, the instrument 7400 or any of the components therein are optionally parts of a surgical system that performs surgical procedures, and which can include a manipulator unit, a series of kinematic linkages, a series of cannulas, or the like. The instrument 7400 (and any of the instruments described herein) can be used in any suitable surgical system, such as the MIRS system 1000 shown and described above. The instrument 7400 includes a proximal mechanical structure, a shaft, and a spacer 7900, which can be similar or the same as the proximal mechanical structure 6700, the shaft 6410 and the spacer 6900 described above. The instrument 7400 includes a distal wrist assembly 7500, a distal end effector 7460, and a set of cables (see FIGS. 32-37 identifying one of the cables as cable 7420). The cables function as tension elements that couple the proximal mechanical structure to the distal wrist assembly 7500 and end effector 7460. In some embodiments, the cables can be constructed from a polymer as described above for the cable 2420. The instrument 7400 is configured such that movement of one or more of the cables produces rotation of the end effector 7460 about a first axis of rotation A1 (see FIG. 24, which functions as a yaw axis, the term yaw is arbitrary), rotation of the wrist assembly 7500 about a second axis of rotation A2 (see FIG. 24, which functions as a pitch axis), a cutting rotation of the tool members of the end effector 7460 about the first axis of rotation A1, or any combination of these movements.

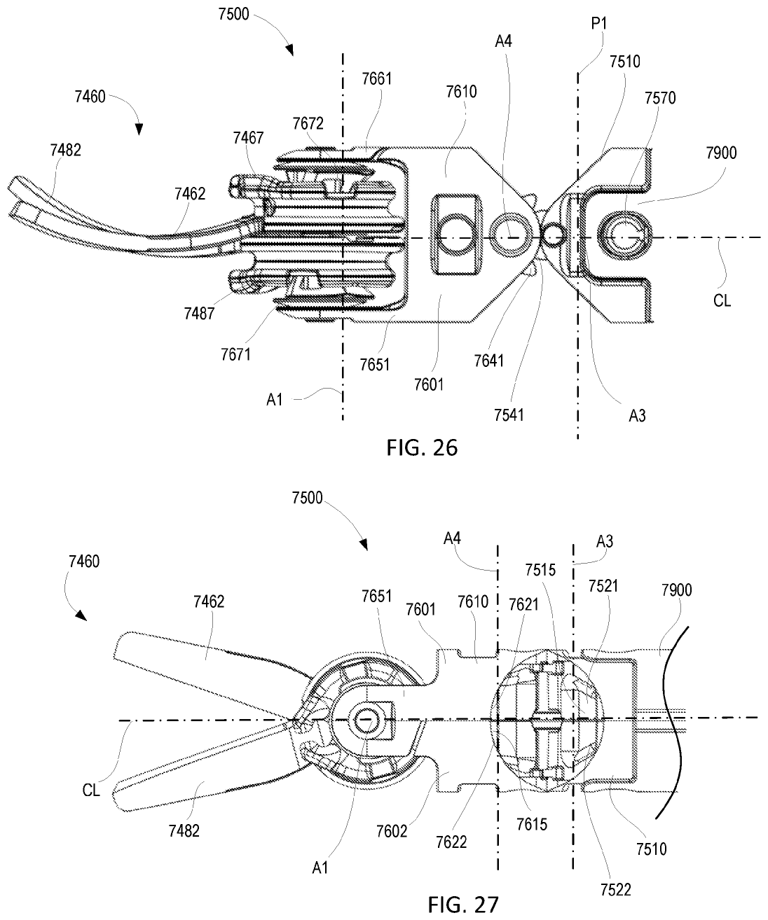

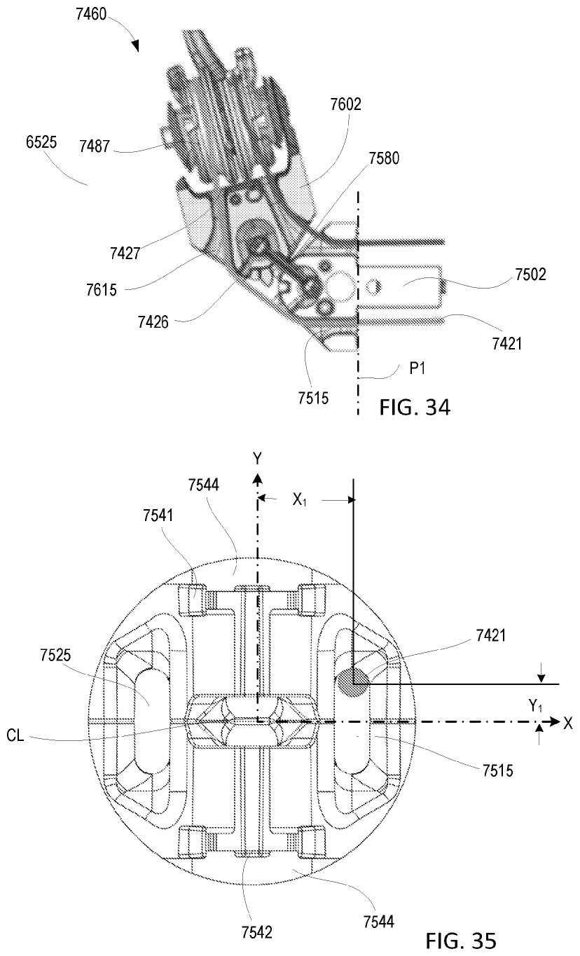

Referring to FIG. 24, the wrist assembly 7500 (also referred to as a joint assembly) includes a first link 7510 (which functions as a proximal wrist link), a second link 7610 (which functions as a distal wrist link) and a connector link 7580. The connector link 7580 is coupled between the first link 7510 and the second link 7610 to form the articulating wrist assembly 7500. The first link 7510 is coupled to the connector link 7580 via a pinned joint such that the first link 7510 is rotatable with reference to the connector link 7580 about a third rotation axis A3 (which functions as a proximal connector link rotation axis; see FIG. 27). The second link 7610 is also coupled to the third link 7515 via a pinned joint such that the second link 7610 is rotatable with reference to the connector link 7580 about a fourth rotation axis A4 (which functions as a distal connector link rotation axis; see FIG. 27). In this manner, the connector link 7580 maintains the coupling between the first link 7510 and the second link 7610 during rotation of the second link 7610 relative to the first link 7510. Further, as described below, the distal end portion 7512 of the first link 7510 includes a joint portion 7540 that is rotatably coupled to a mating joint portion 7640 at the proximal end portion 7611 of the second link 7610. In this manner, the second link 7610 rotates relative to the first link 7510 about the second rotation axis A2 (which functions as the pitch axis, the term pitch is arbitrary). The wrist assembly 7500 defines a longitudinal center line CL (see FIGS. 26, 27). Although the longitudinal center line CL is shown as being linear, when the wrist assembly is moved into different orientations (i.e., when the second link 7610 rotates relative to the first link 7510; see FIG. 34), the longitudinal center line CL can be curved.

The first link 7510 has a proximal portion 7511 and a distal portion 7512. The proximal portion 7511 includes a coupling protrusion that is coupled to the spacer 7900. In some embodiments, some portions of the wrist assembly 7500 are formed with a metallic material and are used in the delivery of electrical energy to the tool members 7462 and 7482. For example, the first link 7510 and the second link 7610 can be formed with a metallic material. Accordingly, as shown, the coupling protrusion of the proximal portion 7511 includes an interior region that forms an electrical connector 7573 configured to receive an electrical wire (not shown) to electrically couple the electrical wire to the first link 7511. The electrical connector 7573 can be a push-in type connector that includes sharp protrusions configured to strip away portions of the insulation from the electrical wire to establish electrical coupling between the wire and the first link 7510.

The distal portion 7512 of the first link 7510 includes a joint portion 7540 that is rotatably coupled to a mating joint portion 7640 of the second link 7610. Specifically, the joint portion 7540 includes a first set of teeth 7541, a second set of teeth 7542 and curved contact surfaces 7544. The first set of teeth 7541 intermesh with the corresponding first set of teeth 7641 on the second link 7610 and the second set of teeth 7542 intermesh with the corresponding second set of teeth 7642 on the second link 7610. When the second link 7610 rotates relative to the first link 7510 (i.e., pitch rotation about the second rotation axis A2), the curved contact surface 7544 are in rolling contact with the corresponding curved contact surfaces 7644 of the second link 7610.

Referring to FIGS. 18 and 19, the first link 7510 includes a discrete first link piece 7501 and a discrete second link piece 7502. Similarly stated, the first link piece 7501 and the second link piece 7502 are constructed as separate pieces and are later coupled together to form the first link 7510. The second link piece 7502 can be coupled to the first link piece 7501 by any suitable mechanism (e.g., an adhesive joint, a weld joint, or a mechanical fastener). Moreover, although described separately herein, in some embodiments, the first link piece 7501 and the second link piece 7502 can have the same configuration. Similarly stated, in some embodiments, the first link piece 7501 and the second link piece 7502 can be substantially identical pieces.

The first link piece 7501 includes a proximal end portion 7503 and a distal end portion 7504. The proximal end portion 7503 includes a portion of the coupling protrusion and the electrical connector 7573. The distal end portion 7504 includes a first set of teeth 7541 and the curved contact surface 7544. The distal end portion 7504 of the first link piece 7501 also includes an opening 7557 that, together with the corresponding opening 7558 of the second link piece 7502, form a proximal connector link receptacle. The distal connector link receptacle (including the opening 7557 and the opening 7558) receives and retains a proximal end 7581 of the connector link 7580. Similarly stated, a side wall of the first link piece 7501 defines the opening 7557 that receives and retains a cylindrical protrusion of the proximal end 7581 of the connector link 7580. The opening 7557 is aligned with the corresponding opening 7558 of the second link piece 7502, and together they define the proximal connector link rotation axis A3.

The first link piece 7501 defines a first channel 7521 and a second channel 7522. The first channel 7521 opposes a corresponding first channel 7531 of the second link piece 7502 and the second channel 7522 opposes a corresponding second channel 7532 of the second link piece 7502. In this manner, when the first link piece 7501 is coupled to the second link piece 7502, the first channel 7521 is aligned with the first channel 7531 to define the first cable guide channel 7515 (see FIGS. 33-35). Similarly, the second channel 7522 is aligned with the second channel 7532 to define the second cable guide channel 7525. The cable guide channels are shaped and contoured to produce the desired path for the cables (e.g., cable 7420 to pass therethrough). Considerations informing the shape of the first cable guide channel 7515 and the second cable guide channel 7525 (and any cable guide channels described herein) include reducing friction losses (from cable movement within the channels), maintaining the cables within the distal boundary (e.g., similar to the distal boundary 6900 described above), minimizing a fleet angle of the cables with respect to their connection to the end effector 7460, and positioning the cables relative to the longitudinal center line CL to maximize the torque that can be applied about one of the axes of rotation of the wrist assembly 7500 or end effector 7460. For example, the cable guide channel 7515 can be shaped to reduce sharp bends, which can reduce friction losses when the cable 7420 is moved therein.

The second link piece 7502 includes a proximal end portion 7505 and a distal end portion 7506. The proximal end portion 7505 includes a portion of the coupling protrusion and the electrical connector 7573. The distal end portion 7506 includes a second set of teeth 7542 and curved contact surface 7544. The distal end portion 7506 also includes the connector link the opening 7558, as described above. The second link piece 7502 defines the first channel 7531 and the second channel 7532, as described above.

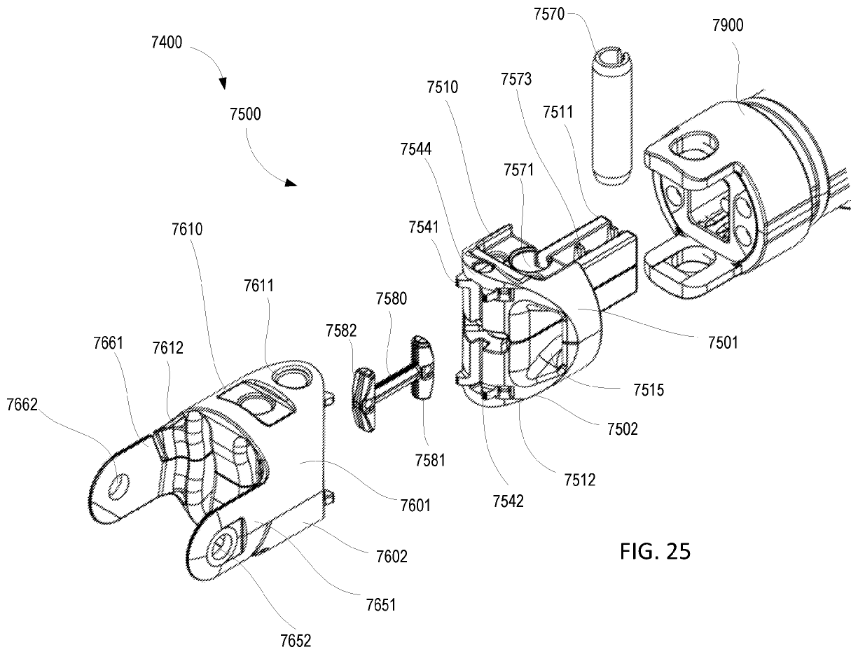

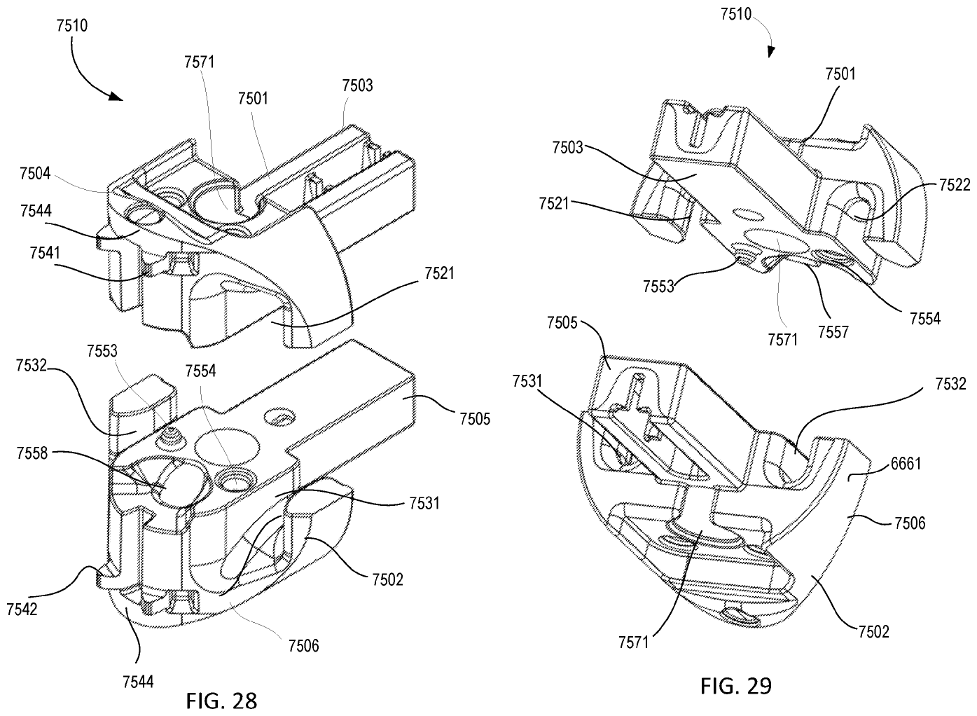

As shown in FIGS. 28 and 29, the first link piece 7501 and the second link piece each include a connection pin 7553 and a connection bore 7554. The connection pin 7553 of the first link piece 7501 is configured to be received within the connection bore 7554 of the second link piece 7502 (see FIG. 28), and vice-versa, to facilitate coupling the first link piece 7501 to the second link piece 7502 to form the first (proximal) link 7510. The first link piece 7501 and the second link piece 7502 each include openings 7571 through which the connection pin 7570 (see FIG. 25) can be inserted to facilitate coupling the first link 7510 to the spacer 7900. When the second link piece 7502 is coupled to the first link piece 7501 to form the first link 7510, the opening 7557 is opposite the opening 7558. Thus, the openings 7557 and 7558 are aligned such that the protrusions of the proximal end 7581 of the connector link 7580 can be coupled therein to allow rotation of the connector link 7580 about the third rotation axis A3.

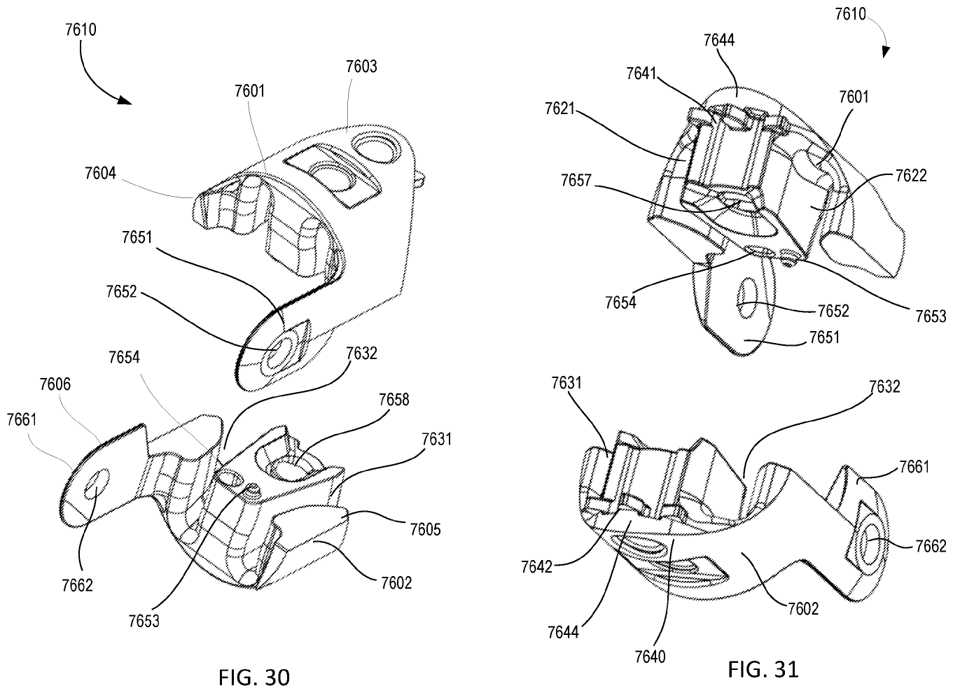

The second (distal) link 7610 has a proximal portion 7611 and a distal portion 7612. The proximal portion 7611 is rotatably coupled to the distal portion 7512 of the first link 7510. As described herein, the second link 7610 rotates relative to the first link 7510 about the second rotation axis A2. The distal portion 7612 is coupled to the end effector 7460. Referring to FIGS. 30 and 31. the second link 7610 includes a discrete first link piece 7601 and a discrete second link piece 7602. Similarly stated, the first link piece 7601 and the second link piece 7602 are constructed as separate pieces and are later coupled together to form the second link 7610. The second link piece 7602 can be coupled to the first link piece 7601 by any suitable mechanism (e.g., an adhesive joint, a weld joint, or a mechanical fastener). Moreover, although described separately herein, in some embodiments, the first link piece 7601 and the second link piece 7602 can have the same configuration. Similarly stated, in some embodiments, the first link piece 7601 and the second link piece 7602 can be substantially identical pieces.

The first link piece 7601 includes a proximal end portion 7603 and a distal end portion 7604. The distal end portion 7604 includes a first clevis ear 7651 that defines an opening 7652. The proximal end portion 7603 includes an opening 7657 that, together with the corresponding opening 7658 of the second link piece 7602, form a distal connector link receptacle. The distal connector link receptacle (including the opening 7657 and the opening 7658) receives and retains a distal end 7582 of the connector link 7580. Similarly stated, a side wall of the first link piece 7601 defines the opening 7657 that receives and retains the cylindrical protrusions the distal end 7582 of the connector link 7580. The opening 7657 is aligned with the corresponding opening 7658 of the second link piece 7602, and together they define the distal connector link rotation axis A4. The proximal end portion 7603 also includes a first set of teeth 7641 and curved contact surface 7544.

The first link piece 7601 defines a first channel 7621 and a second channel 7622. The first channel 7621 opposes a corresponding first channel 7631 of the second link piece 7602 and the second channel 7622 opposes a corresponding second channel 7632 of the second link piece 7602. In this manner, when the first link piece 7601 is coupled to the second link piece 7602, the first channel 7621 is aligned with the first channel 7631 to define the first cable guide channel 7615 (see FIG. 33). Similarly, the second channel 7622 is aligned with the second channel 7632 to define the second cable guide channel 7625.

The second link piece 7602 includes a proximal end portion 7605 and a distal end portion 7606. The distal end portion 7606 includes a first clevis ear 7661 that defines an opening 7662. The proximal end portion 7605 includes an opening 7658 that, together with the corresponding opening 7657 of the first link piece 7601, form a distal connector link receptacle, as described above. The proximal end portion 7605 also includes a first set of teeth 7642 and curved contact surface 7544. The second link piece 7602 defines the first channel 7631 and the second channel 7632, as described above.

As shown in FIGS. 30 and 31, the first link piece 7601 and the second link piece each include a connection pin 7653 and a connection bore 7654. The connection pin 7653 of the first link piece 7601 is configured to be received within the connection bore 7654 of the second link piece 7602, and vice-versa, to facilitate coupling the first link piece 7601 to the second link piece 7602. When the second link piece 7602 is coupled to the first link piece 7601 to form the second link 7610, the second clevis ear 7661 is opposite the first clevis ear 7651. Thus, the openings 7652 and 7662 are aligned such that a pin 7670 can be coupled between the first clevis ear 7651 and the second clevis ear 7661 to allow rotation of the end effector 7460 about a tool member rotation axis A1. Similarly stated, when the second link piece 7602 is coupled to the first link piece 7601, the second clevis ear 7661 is aligned with the first clevis ear 7651 to define the tool member rotation axis A1. Additionally, when the second link piece 7602 is coupled to the first link piece 7601, the opening 7657 is opposite the opening 7658. Thus, the openings 7657 and 7658 are aligned such that the distal end portion 75822 of the connector ink 7580 can be coupled within the openings to allow rotation of the connector link 7580 about the fourth rotation axis A4.

When the second link 7610 is assembled, the end effector 7460 is rotatably coupled to the second link 7610 about the first rotation axis A1. The end effector 7460 includes a first tool member 7462 and a second tool member 7482 that are coupled between the clevis ears 7651 and 7661. The first tool member 7462 includes a contact portion and a pulley portion 7467. The second tool member 7482 includes a contact portion and a pulley portion 7487. The pulley portion 7467 is rotatably coupled to the first clevis ear 7651 and the pulley portion 7487 is rotatably coupled to the second clevis ear 7661 by a pin 7670. The pulley portions each include a contact surface or other structure to which cables can be coupled. In this manner, when actuated the tool members 7462, 7482 can rotate about the tool member rotation axis A1 (which functions as the yaw axis; the term yaw is arbitrary).

As shown in FIGS. 20-22, when the second link 7610 is assembled, the second link defines a first cable guide channel 7615 and a second cable guide channel 7625. The cable guide channels are shaped and contoured to produce the desired path for the cables (e.g., cable 7420 to pass therethrough). Considerations informing the shape of the first cable guide channel 7615 and the second cable guide channel 7625 (and any cable guide channels described herein) include reducing friction losses (from cable movement within the channels), maintaining the cables within a distal boundary, minimizing a fleet angle of the cables with respect to their connection to the end effector 7460, and positioning the cables relative to the longitudinal center line CL to maximize the torque that can be applied about one of the axes of rotation of the wrist assembly 7500 or end effector 7460.

To further illustrate the cable guide channels within the first and second links of the wrist assembly, FIGS. 32-34 show various views of the cable 7420 being routed through the wrist assembly 7500. FIG. 35 shows a side view of the first link 7510 within plane P1 (as identified in FIG. 34). Plane P1 is normal to the longitudinal center line CL of the wrist assembly 7500. The plane P1 is defined by a first dimension (identified as the X-axis in FIG. 35) and a second dimension (identified as the Y-axis in FIG. 35). The Y-axis is parallel to the second rotation axis A2 (i.e., the pitch axis), the third rotation axis A3, and the fourth rotation axis A4. The X-axis is parallel to the first rotation axis A1 (i.e., the yaw axis). As shown in FIG. 15, the first cable guide channel 7515 is shaped and sized so that the first proximal end 7421 of the cable 7420 is offset from the Y-axis by a first X distance X₁ and offset from the X-axis by first Y distance Y₁. Because the Y-axis is parallel to the second rotation axis A2 (i.e., the pitch axis), it is desirable to have a large value for the first distance X₁ to maximize the torque that can be applied by the cables when rotating the second link 7610 about the second rotation axis A2. Additionally, because the cables must be routed within the distal boundary, increasing the first distance X₁ will result in a smaller value for the first distance Y₁. As described in more detail, the cable guide channels through the second link 7610 are configured to route the cable 7420 to reduce the X distance and maximize the Y distance (to increase the torque that can be applied by the cables when rotating the end effector 7460 about the first rotation axis A1 (the yaw axis).

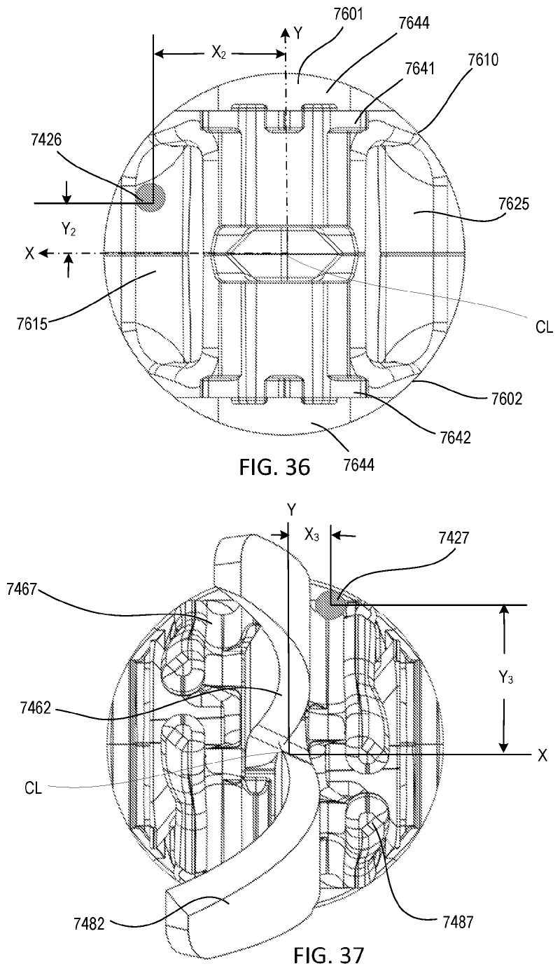

FIG. 36 shows a side view of the second link within plane P2 (as identified in FIG. 33), which can be characterized as an entry plane (or a plane at the entry point where the cables enter the proximal portion 7611 of the second link 7610). FIG. 37 shows a side view of the second link within plane P3 (as identified in FIG. 33), which can be characterized as the exit plane. Planes P2 and P3 are normal to the longitudinal center line CL of the wrist assembly 7600. As with plane P1 described above, the planes P2 and P3 are each defined by a first dimension (identified as the X-axis) and a second dimension (identified as the Y-axis). The cable 7420 includes a first central portion 7426 and a second central portion 7427. The first central portion 7426 of the cable 7420 is between the first proximal portion 7421 and the distal portion 7422, and is the length of cable that is between the exit of the cable guide channel 7515 of the first link 7510 and within the entry point of the cable guide channel 7615 of the second link 7610. Thus, the first central portion 7426 of the cable 7420 is within the cable guide channel 7615 at the second plane P2. The second central portion 7427 of the cable 7420 is between the first proximal portion 7421 and the distal portion 7422, and is the length of cable that exits the cable guide channel 7615 and is coupled to the tool member. Thus, the second central portion 7427 of the cable 7420 is within the cable guide channel 7615 at the third plane P3. As shown in FIGS. 32 and 33, the cable guide channel 7615 is curved in both the X dimension and Y dimension to produce the desired performance.

Specifically, as shown in FIG. 36, the first cable guide channel 7615 is shaped and sized so that the first central portion 7426 of the cable 7420 is offset from the Y-axis by a second X distance X₂ and offset from the X-axis by second Y distance Y₂. Because the Y-axis is parallel to the second rotation axis A2 (i.e., the pitch axis), it is desirable to have a large value for the second distance X₂ to maximize the torque that can be applied by the cables when rotating the second link 7610 about the second rotation axis A2. Additionally, because the cables must be routed within the distal boundary, increasing the second distance X₂ will result in a smaller value for the second distance Y₂. In some embodiments, the second distance X₂ is the same as the first distance X₁. Similarly stated, in some embodiments, the shape and position with respect to the center line CL of the entry portion of the cable guide channel 7615 (i.e., the portion at plane P2) is the same as the shape and position with respect to the center line CL of the entry portion of the cable guide channel 7515 (i.e., the portion at plane P1).

As shown in FIG. 37, the cable guide channel 7615 is curved and sized such that the second central portion 7427 of the cable 7420 is offset from the Y-axis by a third X distance X₃ and offset from the X-axis by third Y distance Y₃. Because the X-axis is parallel to the first rotation axis A1 (i.e., the yaw axis), it is desirable to have a large value for the third distance Y₃ to maximize the torque that can be applied by the cables when rotating the tool member 7482 about the first rotation axis A1. Additionally, because the cables must be routed within the distal boundary, increasing the third distance Y₃ will result in a smaller value for the third distance X₃. Here, the third distance Y₃ is greater than the second distance Y₂ and the third distance X₃ is less than the second distance X₂. The smaller third distance X₃ (which brings the cables more inboard in the X dimension also reduces the fleet angle between the pulley portion 7487 and the second central portion 7427 of the cable.

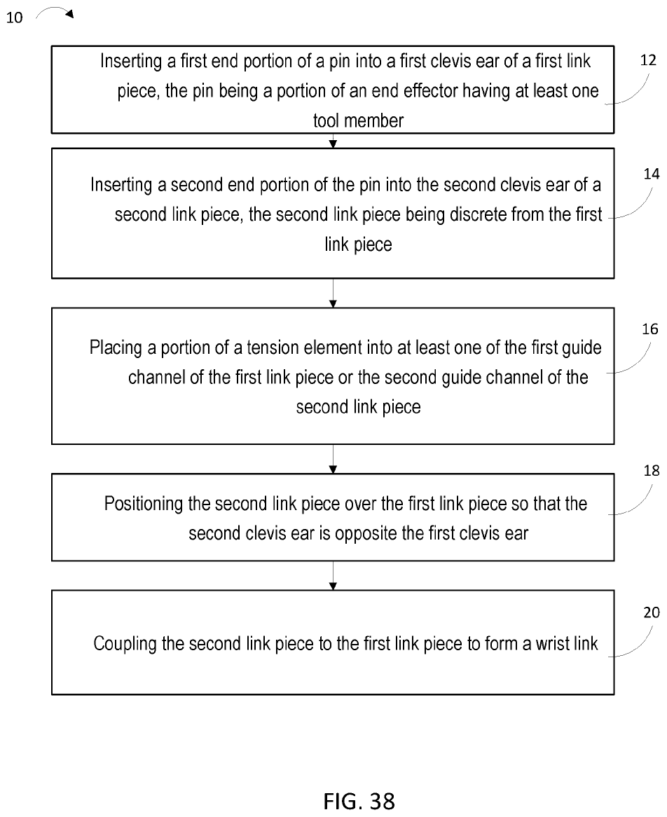

FIG. 38 is a flow chart of a method of assembly a portion of a medical device according to an embodiment. The method can be performed with any of the devices described herein having one or more wrist links being constructed from two (or more) discrete link pieces. Specifically, the method can be used to assemble the medical device 6400 or the medical device 7400 described herein. Such medical devices include a first link piece, a second link piece, a tool member, a pin, and a tension element. The method includes inserting a first end portion of a pin into a first clevis ear of a first link piece, at 12. The first link piece can be any of the first discrete link pieces described herein, such as, for example, the first link piece 6601 or the first link piece 7601. The pin is a portion of an end effector having at least one tool member (e.g., the pin 6670 or 7670 that couple the end effectors 6460 or 7460, respectively). The second end portion of the pin is inserted into the second clevis ear of a second link piece, at 14. The second link piece is discrete from the first link piece and can be any of the second discrete link pieces described herein, such as, for example, the second link piece 6602 or the second link piece 7602.

The method includes placing a portion of a tension element into at least one of the first guide channel of the first link piece or the second guide channel of the second link piece, at 16. The tension element can be a cable, and the method of placing a portion of the cable into a guide channel that is open (i.e., that is not fully surrounded) can make assembly more efficient. The second link piece is then positioned over the first link piece so that the second clevis ear is opposite the first clevis ear, at 18. The second link piece is then coupled to the first link piece to form a wrist link, at 20.

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a series of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the components of a surgical instrument described herein can be constructed from any material, such as medical grade stainless steel, nickel alloys, titanium alloys or the like. Further, any of the links, tool members, tension members, or components described herein can be constructed from multiple pieces that are later joined together. For example, in some embodiments, a link can be constructed by joining together separately constructed components. In other embodiments however, any of the links, tool members, tension members, or components described herein can be monolithically constructed.

In some embodiments, any of the tension elements described herein (including the tension elements 2420, 3420, 4420, 5420, 6420, 7420) can be a cable having a polymeric braided construction. In some embodiments, a distal end portion of any of the tension elements describe herein can include an oil coating. In some embodiments, a distal end portion of any of the tension elements describe here can include a hydrophobic material. In some embodiments, any of the tension elements described herein (including the tension elements 2420, 3420, 4420, 5420, 6420, 7420) can be made from a material having suitable temperature characteristics for use with cauterizing instruments. For example, such materials include liquid crystal polymer (LCP), aramid, para-aramid, and polybenzobisoxazole fiber (PBO). Such materials can provide frictional characteristics that increase the ability for friction coupling and improve holding ability, for example for coupling the tension element to a capstan within a proximal mechanical structure (e.g., the mechanical structure 6710) and/or an end effector. Such ability can also improve slip characteristics (e.g., help prevent the cable from slipping) during operation of the medical device. Such materials may or may not need a coating or other surface treatment to increase the frictional characteristics.

Although the instruments are generally shown as having an axis of rotation of the tool members (e.g., axis A1) that is normal to an axis of rotation of the wrist member (e.g., axis A2), in other embodiments any of the instruments described herein can include a tool member axis of rotation that is offset from the axis of rotation of the wrist assembly by any suitable angle.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A medical device, comprising:
a wrist link, a tool member, and a tension element;
wherein the wrist link includes a discrete first link piece and a discrete second link piece;
wherein the first link piece includes a first clevis ear;
wherein the second link piece includes a second clevis ear;
wherein the second link piece is coupled to the first link piece to position the second clevis ear opposite the first clevis ear and to define a tension element guide channel between the first link piece and the second link piece;
wherein the tool member is coupled to rotate between the first clevis ear and the second clevis ear about a tool member rotation axis;
wherein the tension element is coupled to the tool member and extends from the tool member through the tension element guide channel; and
wherein tension on the tension element urges the tool member to rotate about the tool member rotation axis.

2. The medical device of claim 1, wherein a configuration of the first link piece is the same as a configuration of the second link piece.

3. The medical device of claim 1, wherein:
the wrist link is a distal wrist link;
the medical device includes a proximal wrist link; and
the distal wrist link is coupled to the proximal wrist link and rotates with reference to the proximal wrist link.

4. The medical device of claim 1, wherein:
the wrist link is a distal wrist link;
the medical device includes a proximal wrist link and a connector link;
the connector link includes a distal end and a proximal end;
the distal wrist link is coupled to the distal end of the connector link;
the proximal wrist link is coupled to the proximal end of the connector link; and
the distal wrist link rotates with reference to the connector link about a distal connector link rotation axis;
the connector link rotates with reference to the proximal wrist link about a proximal connector link rotation axis; and
the distal wrist link is in rolling contact with the proximal wrist link as the distal wrist link rotates with reference to the proximal wrist link.

5. The medical device of claim 4, wherein:
the first link piece includes a first connector link receptacle;
the second link piece includes a second connector link receptacle;
the second link piece is coupled to the first link piece to position the second connector link receptacle opposite the first connector link receptacle; and
the distal end of the connector link is rotatably secured to the distal wrist link between the first connector link receptacle and the second connector link receptacle.

6. The medical device of claim 5, wherein:
a first protrusion of the distal end of the connector link is within the first connector link receptacle; and
a second protrusion of the distal end of the connector link is within the second connector link receptacle.

7. The medical device of claim 4, wherein:
the first link piece includes a first connector link protrusion;
the second link piece includes a second connector link protrusion;
the second link piece is coupled to the first link piece to position the second connector link protrusion opposite the first connector link protrusion; and
the distal end of the connector link is rotatably secured to the distal wrist link between the first connector link protrusion and the second connector link protrusion.

8. The medical device of claim 4, wherein:
a longitudinal axis is defined between the distal end of the connector link and the proximal end of the connector link;
the proximal wrist link includes a connector link receptacle;
the connector link receptacle is configured to accept insertion of the proximal end of the connector link at a first orientation of the connector link about the longitudinal axis of the connector link; and
the connector link receptacle is configured to resist withdrawal of the proximal end of the connector link at a second orientation of the connector link about the longitudinal axis of the connector link.

9. The medical device of claim 4, wherein:
the proximal wrist link includes a discrete third link piece and a discrete fourth link piece;
the third link piece includes a third connector link receptacle;
the fourth link piece includes a fourth connector link receptacle;
the fourth link piece is coupled to the third link piece to position the fourth connector link receptacle opposite the third connector link receptacle; and
the proximal end of the connector link is rotatably secured to the proximal wrist link between the third connector link receptacle and the fourth connector link receptacle.

10. The medical device of claim 1, wherein:
the medical device includes a pin;
the pin extends from the first clevis ear, through the tool member, and to the second clevis ear such that the tool member rotates about the pin; and
the pin is under tension.

11. The medical device of claim 10, wherein:
the tool member is a first tool member;
the tension element is a first tension element;
the tension element guide channel is a first tension element guide channel;
the medical device further includes a second tool member and a second tension element;
the second tool member is coupled to rotate between the first clevis ear and the second clevis ear about the tool member rotation axis;
the second tension element is coupled to the second tool member and extends from the second tool member through a second tension element guide channel; and
tension on the second tension element urges the second tool member to rotate about the tool member rotation axis.

12. The medical device of claim 11, wherein:
the medical device includes a first washer and a second washer;
the first washer is positioned between the first clevis ear and the first tool member;
the second washer is positioned between the second clevis ear and the second tool member;
the first washer urges the first tool member towards the second tool member; and
the second washer urges the second tool member towards the first tool member.

13. The medical device of claim 1, wherein the second link piece is coupled to the first link piece by any of an adhesive joint, a weld joint, or a mechanical fastener.

14. A medical device, comprising:
a first link piece, a second link piece discrete from the first link piece, a tool member, and a connector link;
wherein the first link piece includes a distal end portion and a proximal end portion, the distal end portion of the first link piece includes a first clevis ear, and the proximal end portion of the first link piece includes a first connector;
wherein the second link piece includes a distal end portion and a proximal end portion, the distal end portion of the second link piece includes a second clevis ear, and the proximal end portion of the second link piece includes a second connector;
wherein the second link piece is coupled to the first link piece to form a wrist link and to position the second clevis ear opposite the first clevis ear and to position the second connector opposite the first connector;
wherein the tool member is coupled to rotate between the first clevis ear and the second clevis ear about a tool member rotation axis; and
wherein the connector link is coupled to rotate between the first connector and second connector about a connector link rotation axis.

15. The medical device of claim 14, wherein:
the wrist link is a distal wrist link;
the connector link includes a distal end and a proximal end;
the distal end of the connector link is rotatably coupled between the first connector and the second connector;
the medical device includes a proximal wrist link; and
the proximal end of the connector link is rotatably coupled to the proximal wrist link.

16. The medical device of claim 15, wherein:
the distal wrist link rotates with reference to the proximal wrist link; and
the distal wrist link is in rolling contact with the proximal wrist link as the distal wrist link rotates with reference to the proximal wrist link.

17. The medical device of claim 15, wherein:
a longitudinal axis is defined between the distal end of the connector link and the proximal end of the connector link;
the proximal wrist link includes a connector link receptacle;
the connector link receptacle is configured to accept insertion of the proximal end of the connector link at a first orientation of the connector link about the longitudinal axis of the connector link; and
the connector link receptacle is configured to resist withdrawal of the proximal end of the connector link at a second orientation of the connector link about the longitudinal axis of the connector link.

18. The medical device of claim 15, wherein:
the first connector of the distal wrist link is a first connector link receptacle;
the second connector of the distal wrist link is a second connector link receptacle;
the distal end of the connector link is rotatably secured to the distal wrist link between the first connector link receptacle and the second connector link receptacle;
the proximal wrist link includes a discrete third link piece and a discrete fourth link piece;
the third link piece includes a third connector link receptacle;
the fourth link piece includes a fourth connector link receptacle;
the fourth link piece is coupled to the third link piece to position the fourth connector link receptacle opposite the third connector link receptacle; and
the proximal end of the connector link is rotatably secured to the proximal wrist link between the third connector link receptacle and the fourth connector link receptacle.

19. The medical device of claim 14, wherein:
the medical device includes a tension element;
the second link piece is coupled to the first link piece to define a tension element guide channel between the first link piece and the second link piece;
the tension element is coupled to the tool member and extends from the tool member through the tension element guide channel; and
tension on the tension element urges the tool member to rotate about the tool member rotation axis.

20. The medical device of claim 14, wherein the tool member rotation axis is perpendicular to the connector link rotation axis.

21.-35. (canceled)
